(19) United States
(12) Patent Application Publication
(10) Pub. No.: US 2025/0186273 A1
(43) Pub. Date: Jun. 12, 2025

(54) ABSORBENT CORES AND ABSORBENT ARTICLES INCORPORATING THESE

(71) Applicant: The Procter & Gamble Company, Cincinnati, OH (US)

(72) Inventors: Ernesto Gabriel BIANCHI, Oberursel (DE); Michael Josef DIEWALD, Mechernich (DE); Bruno Johannes EHRNSPERGER, Bad Soden am Taunus (DE)

(21) Appl. No.: 18/976,733

(22) Filed: Dec. 11, 2024

Related U.S. Application Data
(60) Provisional application No. 63/608,328, filed on Dec. 11, 2023.

(51) Int. Cl. A61F 13/538

(52) U.S. Cl. CPC A61F 13/538; A61F 2013/5383

(57) ABSTRACT

An absorbent core for personal hygiene articles having a superabsorbent material defining a central area having a first and second recess is provided. Each recess includes an absorbent sideline area having of a series of absorbent material islands separated by a gap when the core is laid flat. The present disclosure is also directed to absorbent articles including the absorbent core, in particular diapers.

### CROSS REFERENCE TO RELATED APPLICATION

This application claims the benefit, under 35 U.S.C. 119(e), of U.S. Provisional Application No. 63/608,328, filed Dec. 11, 2023, the substance of which is incorporated herein by reference.

### FIELD OF THE INVENTION

The invention relates to absorbent cores as well as absorbent articles incorporating these absorbent cores, such as baby diapers and adult incontinence products.

### BACKGROUND OF THE INVENTION

Modern personal hygiene absorbent articles such as baby diapers and adult incontinence diapers are manufactured by converting multiple layers and components at high speed. These absorbent articles comprise a chassis at least formed by a fluid-permeable topsheet on the wearer-facing side of the article, a fluid-impermeable backsheet on the garment-facing side of the article, and an absorbent core disposed in-between these two layers. The absorbent core comprises an absorbent material that may be a mixture of cellulose fibers and superabsorbent polymer particles (“SAP”). Over the years, the relative SAP content has increased while the cellulose fibers content has reduced, thus providing thinner absorbent cores. Absorbent cores without cellulose fibers, so called airfelt-free cores, have also been recently proposed. The absorbent article also typically comprises at least one acquisition-distribution layer between the topsheet and the absorbent core.

Leakage prevention is primarily provided by the permanent immobilization of urine in the absorbent core. However, modern personal hygiene absorbent articles comprise other components to further improve the performance of the articles such as a gasketing system. Gasketing systems typically comprise a pair of inner leg cuffs and a pair of outer leg cuffs that support the absorbent core near the body discharge area. The inner leg cuffs (also referred to as barrier leg cuffs) have a proximal edge joined to the chassis of the article and an elasticized distal edge that can stand up from the chassis to fit tight against the wearer's skin. The outer leg cuffs (also referred to as gasketing cuffs) are also elasticized but are formed in the same plane as the chassis of the articles. Such gasketing systems improve the fit of the article and provide a temporary storage for urine until the fluid is absorbed by the absorbent material. Absorbent cores can absorb many times their weight of urine. One of the key limiting factor is the kinetic of absorption.

Absorbent articles tend to sag down in the crotch region due to the weight of the fluid absorbed. Sagging may cause the inner or outer leg cuffs to lose contact along the thighs of the wearer, thus increasing the possibility of leakages. While the leg cuffs normally maintain contact and fit, efficiency is limited for heavily loaded diapers especially if the diaper was not put in place correctly or was displaced out of position by the wearer. Sagging is a common cause for side leakages of absorbent articles such as baby diapers.

Attempts have been made to reduce this problem by increasing the height and width of the inner and outer cuffs respectively, however this requires wider chassis designs at crotch and the additional materials can negatively impact fit and cost.

Absorbent cores having a narrow width at crotch can solve the tension between higher gasketing systems (inner and outer cuffs) and better fit between the legs. While having a narrow width at crotch cores increases the effective cuff heights, it also significantly reduces the absorbent material available in the crotch region. These cores can also limit the dimensions of the acquisition-distribution layer, which has a width designed to match or being narrower than the width of the absorbent core. This reduced width of the acquisition-distribution layer increases the likelihood of free urine presence at the surface of the absorbent article. This in turn increases the chances that urine will flow over the leg cuffs and away from the absorbent structures, for example if the wearer is laying on the side.

Absorbent cores comprising a central absorbent portion and two absorbent side portions separated by folding guides have also been suggested for providing an improved fit and reduced leakage.

Despite the improvements suggested in the prior art, there is a continuous need for improving dry and wet fit, wearing comfort, ease of application and fluid handling properties, including fluid acquisition without compromising leakage especially at the legs, of absorbent articles while keeping the cost of production as low as possible. The present invention addresses these multiple requirements.

### SUMMARY OF THE INVENTION

In an aspect, the invention relates to an absorbent core extending in a longitudinal direction and a transversal direction. The absorbent core has a longitudinal axis extending in the longitudinal direction. The absorbent core comprises a core wrap having a top layer and a bottom layer, and an absorbent material disposed between the core wrap's top layer and bottom layer. The absorbent material comprises superabsorbent polymer particles and optionally cellulose fibers. The absorbent material defines an absorbent material area that comprises a non-rectangular central area having a front edge, a back edge and two longitudinally-extending side edges joining the front edge to the back edge. The two longitudinally-extending side edges define at least along a section of their length a narrow crotch portion comprising absorbent material, for example the two longitudinally-extending side edges may be inwardly curved towards the longitudinal axis along the narrow crotch portion. The narrow crotch portion thus has a smaller width than the front portion and/or back portion of the central area. A first recess and a second recess are present laterally outwardly of the narrow crotch portion, the recesses being substantially free of absorbent material except for a first absorbent material sideline area and a second absorbent material sideline area present in respectively the first recess and second recess, in particular along the outward longitudinal edges of the recesses.

Each sideline area may have a length which is from about 25% to about 70% of the length of the central area.

Each sideline area may comprise a plurality of substantially longitudinally-aligned islands of absorbent material, each island being separated from adjacent islands by a gap which is free of absorbent material when the absorbent core is laid flat.

The absorbent core may also comprise one or more channel-forming area(s). The channel-forming area(s) is/are absorbent material-free area(s) within the central area where the top layer of the core wrap is bonded to the bottom layer of the core wrap. When the absorbent material surrounding the channel-forming area(s) absorbs a fluid such as urine and swells, three-dimensional channels are formed along the length of the material-free area(s).

The absorbent material may also be free of cellulosic fibers.

These different features of the invention may be combined in an inventive core according to the invention.

The invention also relates to an absorbent article comprising an absorbent core as described above. Such an absorbent article may typically comprise a topsheet, a backsheet, optionally an acquisition and/or distribution layer and a gasketing system. The absorbent article may be in the form of a taped diaper (for baby or adult incontinence), a pant diaper or an insert to be placed in an underwear-like cover material.

### BRIEF DESCRIPTION OF THE DRAWINGS

FIG. 1 is a schematic top view of an absorbent core according to the invention with the top layer of the core wrap partially removed;

FIG. 2 is a transversal cross-section of the core of FIG. 1 (the thickness of the absorbent layer is exaggerated);

FIG. 3a is a schematic perspective representation of the core of FIG. 1 in a bent shape as when worn in an absorbent article;

FIG. 3b is a schematic top view of the core of FIG. 3a;

FIG. 4 is a transversal cross-section of the core of FIG. 1 with the absorbent sideline areas raised;

FIG. 5 is a top view of a flattened-out exemplary taped diaper comprising the core of FIG. 1, with some layers partially removed;

FIG. 6 is a transversal schematic representation of a cross-section of the diaper of FIG. 5, the thickness of the absorbent core and acquisition layer are exaggerated;

FIG. 7 is a schematic representation of a cross-section of the article of FIG. 5 when worn between the thighs of a wearer;

FIG. 8 is a schematic top view of an alternative absorbent core according to the invention comprising two channel-forming areas;

FIG. 9 is a transversal cross-section of the core of FIG. 8 (the thickness of the absorbent layer is exaggerated);

FIG. 9a is a transversal cross-section of the core of FIG. 8 with the different layers in exploded view;

FIG. 10 is a top view of a flattened-out exemplary taped diaper comprising the core of FIG. 8;

FIG. 11 is a schematic representation of a cross-section of the article of FIG. 8 when worn between the thighs of a wearer;

FIG. 12 is a top view of a flattened-out exemplary alternative taped diaper comprising the core of FIG. 8 and an acquisition layer and a distribution layer;

FIG. 13 is a transversal schematic representation of a cross-section of the diaper of FIG. 12;

FIG. 14 is a schematic representation of a cross-section of the alternative article of FIG. 12 comprising an acquisition layer and a distribution layer, when worn between the thighs of a wearer;

FIG. 15 is a schematic representation of an alternative of FIG. 14, wherein the distribution layer comprises a pair of forming guides;

FIGS. 16-19 are schematic top views of alternative absorbent cores according to the invention with the top layer of the core wrap partially removed;

### DETAILED DESCRIPTION OF THE INVENTION

### Introduction

As used herein, the terms “comprise(s)” and “comprising” are open-ended; each specifies the presence of the feature that follows, e.g. a component, but does not preclude the presence of other features, e.g. elements, steps, components known in the art or disclosed herein. These terms based on the verb “comprise” should be read as encompassing the narrower terms “consisting essentially of” which excludes any element, step or ingredient not mentioned which materially affect the way the feature performs its function, and the term “consisting of”′ which excludes any element, step, or ingredient not specified. Any preferred, advantageous or exemplary embodiments described below are not limiting the scope of the claims, unless specifically indicated to do so. The words “typically”, “normally”, “preferably”, “advantageously”, “in particular” and the likes also qualify features which are not intended to limit the scope of the claims, unless specifically indicated to do so. Any feature or component described herein in relation with one embodiment may be combined with another feature or component of another embodiment unless indicated otherwise.

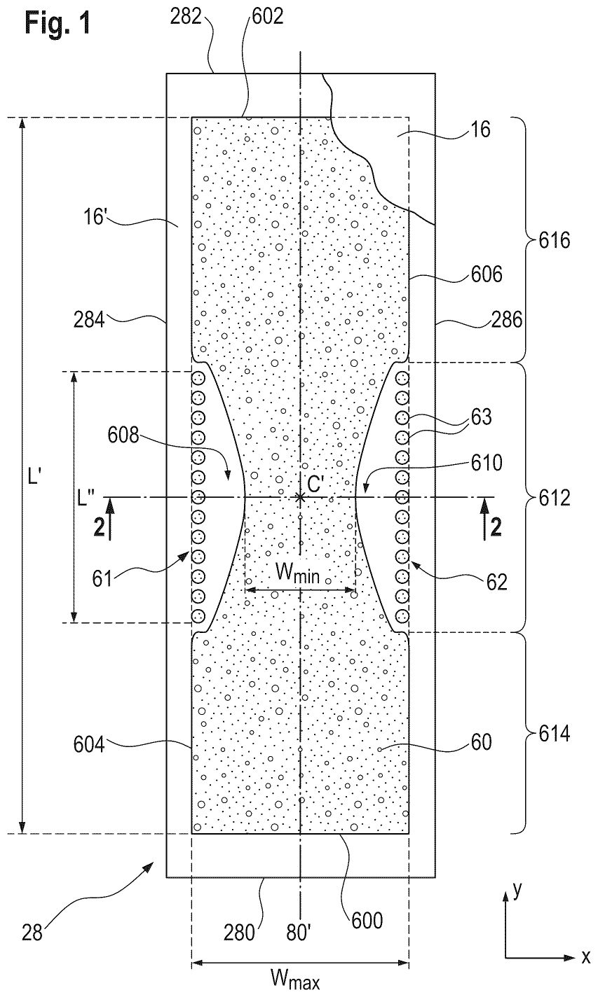

Unless indicated otherwise, the description and claims refer to the absorbent article, absorbent core or component thereof before use (i.e. dry, and not loaded with a fluid) and conditioned at least 24 hours at 21° C.+/−2° C. and 50+/−20% Relative Humidity (RH) and in a flat state as shown for example on FIG. 1.

A “nonwoven web” or “nonwoven” as used herein means a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms such as short fibers (known as staple, or chopped), continuous single fibers (filaments or monofilaments), untwisted bundles of continuous filaments (tow), and twisted bundles of continuous filaments (yarn). Nonwoven webs can be formed by many processes such as meltblowing, spunbonding, solvent spinning, electrospinning, carding and airlaying. The basis weight of nonwoven webs is usually expressed in grams per square meter (g/m² or gsm).

The absorbent articles of the invention and their components will now be discussed generally and with exemplary reference to the Figures and the numerals referred to in these Figures for illustration purpose. These examples are not intended to limit the scope of the claims unless specifically indicated.

### General Description of the Absorbent Core 28

As used herein, the term “absorbent core” refers to an absorbent material in a core wrap2, and which can be integrated in a personal hygiene absorbent article. The absorbent core is typically an individual component, which is converted with other components such as a topsheet and a backsheet to form the absorbent article in a converting line. The core wrap material is typically a nonwoven or tissue paper material defining a top layer and bottom layer, which may be sealed transversally and longitudinally, but it is not excluded that the absorbent core may be formed directly on the backsheet, the topsheet or another layer, in which case the core wrap may be at least partially formed by one of these layers. The term “absorbent core” as used herein does not include a separate liquid management layer that temporarily acquires and distribute the fluid, and which is also typically present in an absorbent article. The absorbent core is typically the component of an absorbent article that has the most absorbent capacity of all the components of the absorbent article, and which comprises all, or at least the majority of, superabsorbent polymer (SAP). The core may consist essentially of, or consist of, the core wrap, the absorbent material and optionally adhesives. The terms “absorbent core” and “core” are used interchangeably herein.

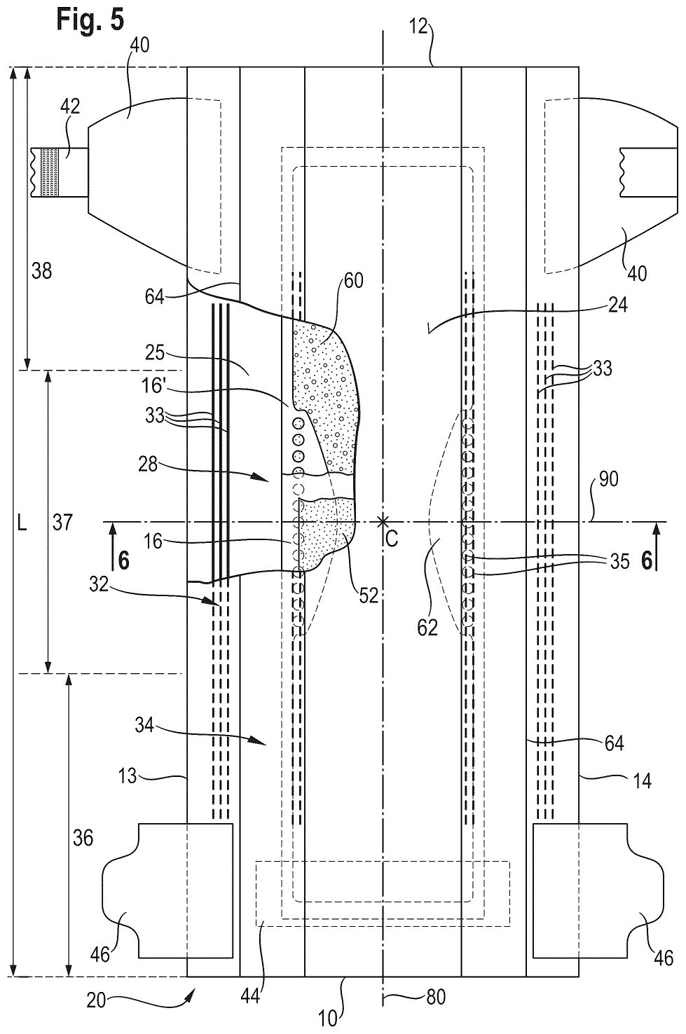

The absorbent cores of the invention are substantially planar. By substantially planar, it is meant that the absorbent core can be laid flat on a planar surface, as represented in FIG. 1. The absorbent cores may also be typically thin and conformable, so that they can also be laid on a curved surface for example a drum during the making process, or stored and handled as a continuous roll of stock material before being converted into an absorbent article. The thickness of the absorbent core (before use) is relatively small compared to dimensions in the transversal direction (x) and the longitudinal direction (y). These directions also correspond respectively to the transversal direction and longitudinal direction respectively of the article in which the absorbent core can be integrated. Unless otherwise indicated, dimensions and areas disclosed herein apply and are measured on the core in this flat-out configuration as shown in FIG. 1. The same applies to an absorbent article, as exemplarily represented in FIG. 5 as a taped diaper, in which the absorbent core may be integrated.

The absorbent cores and articles of the invention are now discussed with reference to the Figures and the numerals referred to in these Figures. These examples are not intended to limit the scope of the claims unless specifically indicated.

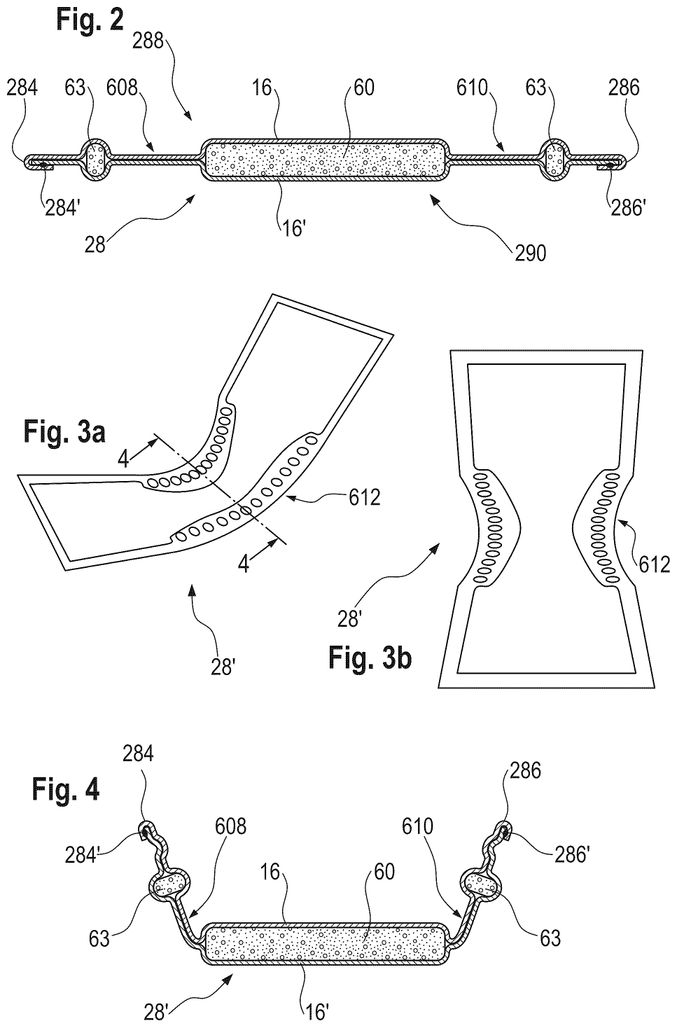

As illustrated in FIGS. 1-2, the absorbent cores 28 of the invention may comprise a front edge 280, a back edge 282 and two longitudinal side edges 284, 286 joining the front edge and the back edge. The front edge of the core is intended to be placed towards the front of the absorbent article in which the core is or will be integrated. The front edge 280 and the back edge 282 of the core are typically shorter than the longitudinal side edges 284, 286 of the core. The absorbent core also comprises a top side 288 and a bottom side 290. The top side of the core is placed or intended to be placed towards the wearer-facing side (topsheet 24) of an absorbent article and the bottom side is the side placed or intended to be placed towards the garment-facing side (backsheet 25) in the absorbent article.

The absorbent core notionally comprises a longitudinal axis 80′ parallel to the longitudinal direction y and extending from the front edge 280 to the back edge 282, and dividing the core in two substantially symmetrical halves relative to this axis. The absorbent core may notionally comprise a crotch point C′ on the longitudinal axis 80′, which corresponds to the point on the longitudinal axis 80′ where the central area 60 of the core has its minimum width Wmin (discussed below).

### Core Wrap

The core wrap may be any usual material suitable for receiving and containing the absorbent material. Typical core wrap materials may be selected from nonwovens, paper, tissues, films, wovens, or laminate of any of these. The core wrap material may in particular be a nonwoven, such as a carded nonwoven, a spunbond nonwoven (“S”) or a meltblown nonwoven (“M”), and laminates of any of these. For example, spunmelt polypropylene nonwovens are suitable, in particular those having a SMS laminate structure, or SMMS, or SSMMS, structure. A core wrap layer may typically have a basis weight of from about 5 gsm to about 15 gsm. Suitable materials are for example disclosed in U.S. Pat. No. 7,744,576, US2011/0268932A1, US2011/0319848A1 and US2011/0250413A1. Nonwoven materials provided from synthetic fibers may be used, such as PE, PET and in particular PP. Higher basis weight material, in particular having a basis weight of at least 40 gsm, may also be used if the core wrap layer has other function, such as a masking layer for the bottom layer of the core wrap.

The core wrap may, as shown in the cross-sectional view of FIG. 2, comprise a top layer 16 forming the top side 288 of the core and a bottom layer 16′ forming the bottom side 290 of the core. The top layer may advantageously be more hydrophilic than the bottom layer, for example the top layer may be treated with a wetting agent. Alternatively, it is also known in the art to make a core wrap out of a single substrate, or to use the backsheet or topsheet as substrate to directly, partially or completely form the core wrap. When two discrete substrates are used, they may be formed with a C-wrap scal 284′, 286′ along each longitudinal side edges 284, 286 of the absorbent core. The longitudinal C-wrap seals may be adhesively formed, as in FIG. 2, but any other seals or core wrap construction known in the art may be used in the present invention. The core wrap may optionally also have a front end seal and a back end seal. Examples of core wrap construction are further for example detailed in WO2014/093310. The core wrap is not considered as absorbent material for the purpose of calculating the percentage of SAP in the absorbent core.

### Absorbent Material Area

The absorbent material defines an absorbent material area within the core wrap, as seen from the top of the core, as exemplarily illustrated in FIG. 1. The absorbent material area is substantially symmetrical relative to the longitudinal axis 80′, taking into account normal process deviation.

The absorbent material area comprises at least a longitudinally-extending, non-rectangular, central area 60, which overlaps at least a portion of the longitudinal axis 80′. The central area 60 has a front edge 600, a back edge 602 and two longitudinally-extending side edges 604, 606 joining the front edge 600 to the back edge 602 on each side of the central area 60. The central area 60 further comprises a pair of recesses 608, 610 disposed symmetrically relative to the longitudinal axis 80′. The recesses 608, 610 are formed by the two longitudinally-extending side edges 604, 606 of the central area 60 significantly narrowing along a portion of the centra area of a certain length disposed between the front edge 600 and the back edge 602. The narrow portion of the central area 60 corresponding longitudinally to the recesses 608, 610 is hereby defined as the crotch portion 612 of the central area 60. The portion of the central area 60 extending from the back edge 602 to the crotch portion 612 is hereby defined as the central area's back portion 616 and the portion of the central area 60 extending from the front edge 600 to the crotch portion 612 is hereby defined as the central area's front portion 614.

The crotch portion 612 has a minimum width Wmin, which is smaller than the maximum width Wmax of the central area 60 in the front portion 614 or the back portion 616. The central area 60 maximum width Wmax may be the width of the front edge 600 and/or the width of the back edge 602. As represented, the front portion 614 and the back portion 616 may have a substantially constant width along their respective length, and this substantially constant width which may be the same for both portions, but it is not excluded that the front portion may be generally less wide than the back portion or vice versa.

The longitudinally-extending side edges 604, 606 of the absorbent material's central area 60 may be substantially straight outside the crotch portion 612. The longitudinally-extending side edges 604, 606 may in particular be straight and parallel to the longitudinal direction in the front portion 614 and/or the back portion 616 of the centra area 60. The longitudinally-extending side edges 604, 606 may be substantially straight and optionally parallel to the longitudinal direction y in both the front portion 614 and the back portion 616 of the central area (as illustrated in FIG. 1).

The central area 60 has a length L′ measured along the longitudinal axis 80′. The central area 60 may be unitary, as represented, but it may be optionally segmented in sub-areas for example separated by transversally-orientated folding guides to provide more flexibility in the longitudinal direction. The length L′ of the central area 60 may be about the same as length of the absorbent core 28 (including the core wrap), in particular L′ may be from 90% to 100% of the length of the absorbent core 28 as a whole. The central area 60 also has a maximum width Wmax, which may be the width of the front edge 600 and/or back edge 602 of the central area 60.

The central area 60 is non-rectangular and comprises two substantially absorbent material-free recesses 608, 610 disposed at a position intermediate between the front edge 600 and the back edge 602 of the central area 60. The overall shape of the central area 60 may in particular be a dog-bone or hour-glass shape, when viewed from the top of the core laid flat as illustrated in FIG. 1. The central area 60 has a minimum width Wmin within the recesses, thus at a longitudinal position intermediate between the front edge 600 and back edge 602 of the central area 60. The central area's minimum width Wmin may range from 10% to 80% of the central area's maximum width Wmax, in particular from 20% to 70%, or from 30% to 56% of the central area's maximum width Wmax. The central area 60 may have a constant width in the front and back portions 614, 616 outside the crotch portion 612, but other configurations are possible, for example the width thereof may continuously expand towards the front and/or back edges of the core. The combined area of the two recesses 608, 610 may typically have an area in range of from 5% to 30% of the trapezoid defined by the front edge 600 and the back edge 602 of the central area (e.g. the rectangle having the dimensions L′×Wmax, or the area of a corresponding trapeze if the front edge and the back edge have different widths).

The absorbent material area further comprises a first sideline area 61 and a second sideline area 62, disposed respectively in the first recess and second recess, on either side of the longitudinal axis 80′. The sideline areas are longitudinally-extending and are disposed along the outward edges of the recesses 61, 62. The sidelines areas may be generally longitudinally aligned with the longitudinally-extending side edges 604, 606 in the front portion 614 and the back portion 616 of the central area 60. The sidelines areas 61, 61 may be aligned in the longitudinal direction as illustrated in FIG. 1 and following but other configurations are also possible.

Each sideline area 61, 62 may be comprised of a plurality of absorbent material islands 63, each island being separated from the adjacent islands by a gap which is free of absorbent material (as seen from the top, when the absorbent core is dry and laid flat, as illustrated in FIG. 1). The plurality of islands 63 forming each sideline area may be generally longitudinally-aligned, in particular they may be aligned parallel to the longitudinal direction y. The islands 63 may also be aligned in the continuity of the longitudinally-extending side edges 604, 606 of the central area outside the recesses, as illustrated in FIG. 1, so that the absorbent material area as a whole has a generally rectangular perimeter when considering the absorbent material sideline areas 61 and 62 as a continuation of the longitudinal side edge 604, 606 of the central area 60 in the crotch portion 612.

The dimensions of the islands and the gap between adjacent islands may vary or be the same for each island and gap. The average diameter of the islands may typically range from 2 mm to 10 mm, for example about 5 mm. The average gap between adjacent islands may be typically have about the same dimension as the island's diameter, typically ranging from 2 mm to 10 mm, for example or about 5 mm. The island may be substantially circular, but other shapes are possible. For non-circular island, the diameter is measured as the longest distance measurable going through the center of each island. It is understood that even modern manufacturing requirement do not allow to deposit perfectly circular islands of absorbent material at the high speed required. The substantially circular islands may thus also be described as blots or dots.

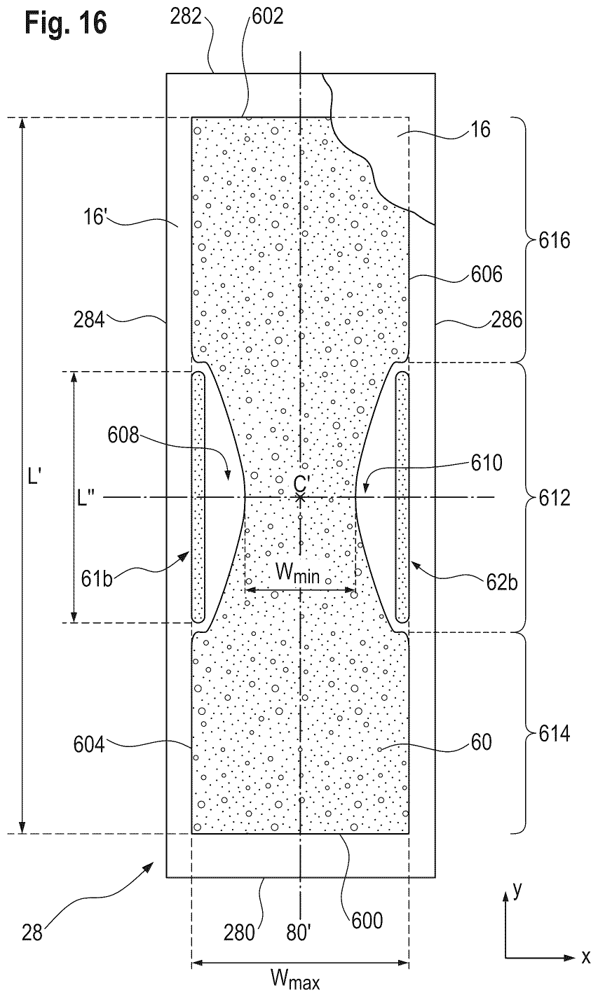

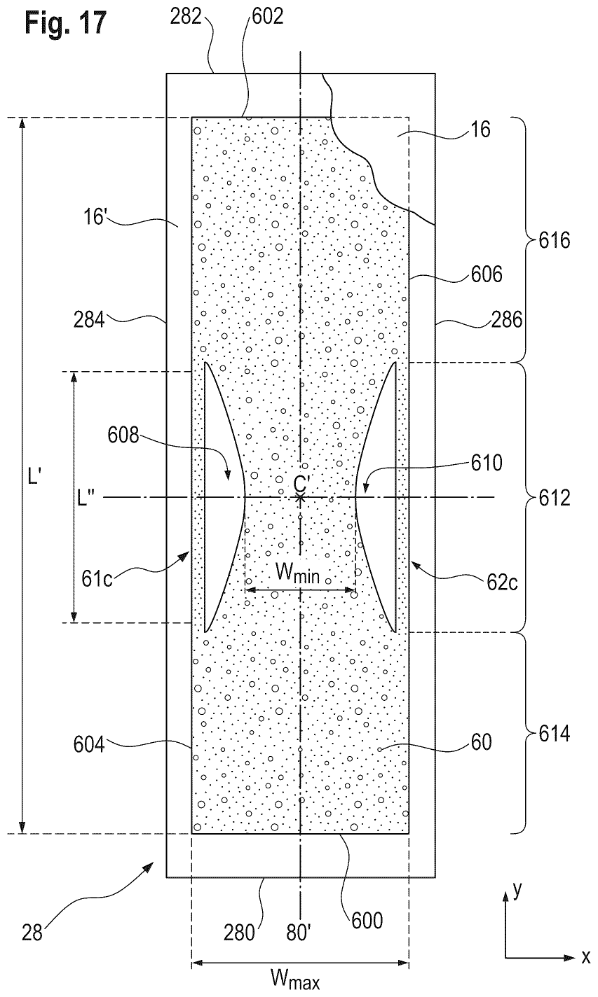

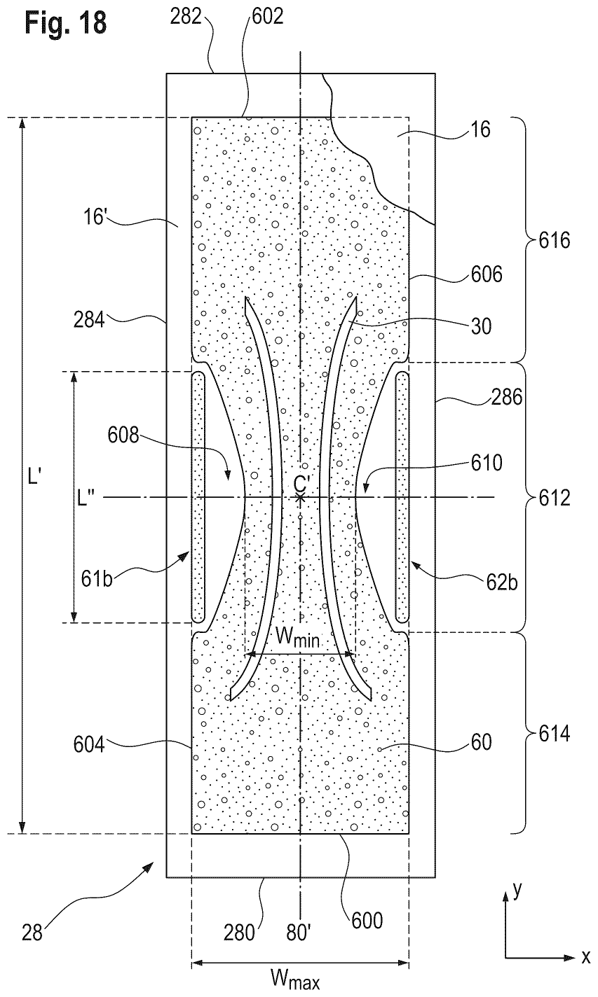

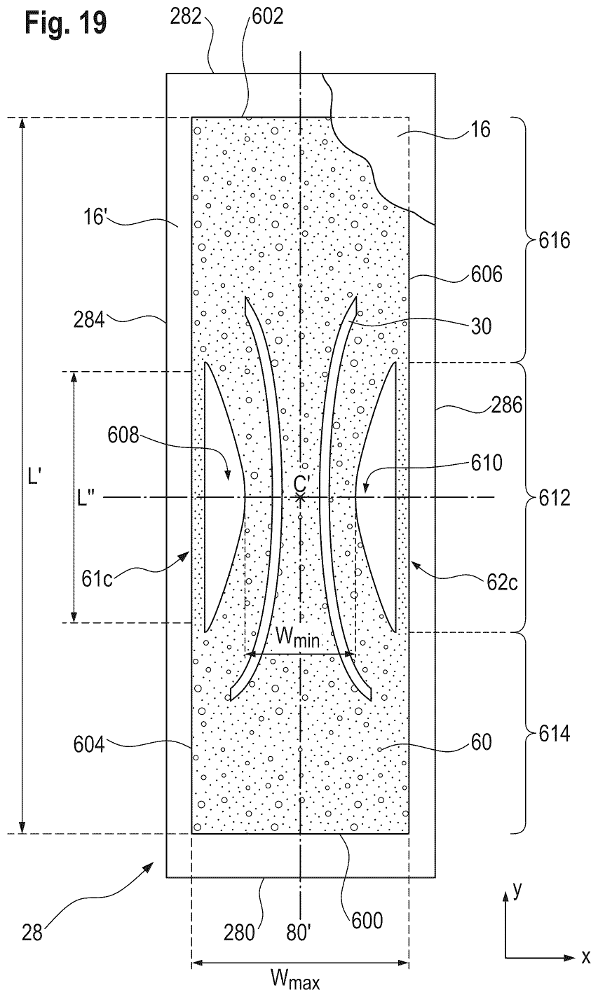

Alternatively, each of the sideline areas may each be formed as a continuous area 61b, 62b unconnected to the central layer 60, as illustrated in FIG. 16 and FIG. 18. In another alternative, the sideline areas 61c, 62c, as illustrated in FIG. 17 or 19, may be formed as a continuous area 61b, 61c connected at one or both of their extremities to the central layer 60. The s

The length L″ of each sideline area 61, 62 is measured in the longitudinal direction y. When the sideline areas are formed by several islands of absorbent material, as illustrated in FIG. 1, the length L″ is measured from the first island to the last island. The length L″ may be ranging from about 25% to about 70%, or from 30% to 60%, of the length L′ of the absorbent material central area 60. The islands may be substantially aligned with the longitudinal direction y, for example perfectly aligned as illustrated in FIG. 1, or aligned at an angle (e.g. up to 40 degree relative to the longitudinal direction), or distributed alternatively on either side of a line parallel to the longitudinal direction y.

The sidelines areas are longitudinally-extending, so that their length is a multiple of their width. Typically the width of the sideline areas may by ranging from 2 mm to 2 cm, in particular from 3 mm to 10 mm. The length to width ratio of the sideline areas may typically range from 2 to 20, in particular from 4 to 14.

Providing absorbent sideline areas has several benefits. Firstly, the sideline areas 61, 62 provide absorbent capacity within the otherwise absorbent material-free recesses 608, 610 in the crotch potion 612. Secondly, the sidelines areas also reduce the risk of side leakage when the acquisition layer 52 or distribution layer 54 extend transversally outwardly of the central portion over the recesses 608, 610. The acquisition layer 52 and/or distribution layer 54 may thus have a width up to about equal to the width W1 of the absorbent material area, with a reduced risk of side leakage at the crotch portion 612. The sideline areas can act as a barrier for a fluid running sideways.

Thirdly, when the absorbent sideline areas are present and comprise islands 63 separated by gaps free of absorbent material, the distance between adjacent islands may be reduced (up to contact between the islands), thus providing tighter sidewalls of protection against side leakages. The gaps between the absorbent material islands 63 are flexible as the core wrap material present therein can easily deform. When the absorbent core assumes a basin shape in use, the absorbent material islands can come closer to each other and the gaps between adjacent islands diminish. In some cases, the islands may contact each other when the article is worn by the wearer. This increases the fluid barrier properties of the side lines when the article in use, especially when the absorbent material in the islands 63 starts absorbing urine and expand, further closing the gaps between the islands of absorbent material 63.

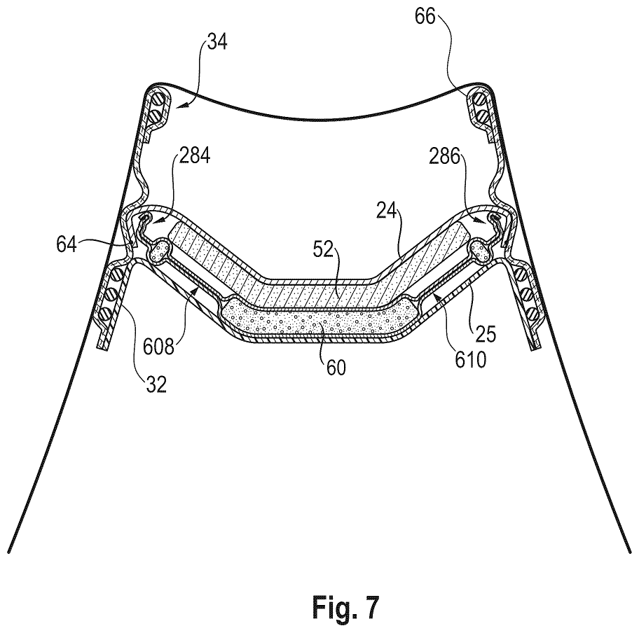

Lastly, as illustrated in FIGS. 3a-b, the crotch potion 612 is particularly flexible due to the recesses 608, 610 being free of absorbent material apart from the sideline areas 61, 62. This flexibility helps the absorbent core to assume a basin shape when the front portion and back portion of the core are bending upwards. The longitudinal side edges 284, 286 of the core can also be more easily folded upwardly, with the absorbent material-free recesses 608, 610 acting as hinges when the article is worn, as illustrated in FIG. 4 and FIG. 7. This flexibility on the crotch portion of the core can help the barrier leg cuffs to maintain a contact with the wearer's thighs. The longitudinal edges 284, 286 of the core can reach a higher position of the thighs of the wearer, thus increasing the effective cuff heights of the articles in which the absorbent core is integrated.

The longitudinal side edges 604, 606 of the central area 60 and/or the sideline areas 61,62 may extend widthwise to the longitudinal side edges 284, 286 of the core wrap. The front edge 600 and the back edge 602 of the central area 60 may on the other hand be optionally spaced from the front edge 280 and back edge 282 of the core wrap respectively, as illustrated in FIG. 1, so that the core wrap may comprise a front end seal and a back end seal in the terminal absorbent material-free areas thus formed at the front and back edge of the core. The top layer and bottom layer of the core wrap may be bonded in these terminal areas to prevent absorbent material from escaping the core wrap from its front edge or back edge. The front edge 600 of the central area 60 may be coterminous or adjacent or separated from the front edge 280 of the absorbent core. The back edge 602 of the central area 60 may be coterminous or adjacent or separated from the back edge 282 of the absorbent core. The core wrap may optionally be sealed along any of its sides, or its whole periphery. The core wrap may in particular comprise side seals 284′, 286′ along the longitudinally-extending side edges of the core, which may be sandwich seals or C-wrap seals as illustrated in FIG. 2. However, it is also possible that there is no core wrap seal on one or more sides of the absorbent core, for example no seals along the front edge and back edge of the core.

### Absorbent Material

The absorbent core comprises an absorbent material that can absorb urine or other bodily exudates such as menstrual fluid. The absorbent material comprises superabsorbent polymer particles, optionally mixed with cellulose fibers. The term “superabsorbent polymer” (herein abbreviated as “SAP”) refers to absorbent materials that can absorb at least 15 times their weight of an aqueous 0.9% saline solution as measured using the Centrifuge Retention Capacity (CRC) test (EDANA method WSP 241.2-05E). The SAP may in particular have a CRC value of from 20 g/g to 50 g/g, or from 25 g/g to 40 g/g. The absorbent material comprises SAP particles but other forms are also possible, such as absorbent foam or fibers. SAP are typically cross-linked acrylate polymeric material. Further detailed examples of absorbent material, in particular SAP, are disclosed in WO2014/093310 (Ehrnsperger). The absorbent material may also comprise or consist of SAP particles that require a time to reach an uptake of 20 g/g (T20) of less than 240s as measured according to the K(t) test method described in WO2012/174026A1 (Ehrnsperger). The SAP particles used may have a permeability at equilibrium expressed as UPM (Urine Permeability Measurement) value of at least 10×10⁻⁷ (cm³·s)/g, in particular at least 15×10⁻⁷ (cm³·s)/g, or at least 20×10⁻⁷ (cm³·s)/g, or from 10 to 50×10⁻⁷ (cm³·s)/g, as measured by the test method indicated in WO2012/174026A1.

The absorbent material may be a blend of cellulose fibers and superabsorbent polymer particles, typically with the percentage of SAP ranging from about 50% to about 75% by total weight of the absorbent material. The absorbent material advantageously comprises a high proportion of superabsorbent polymer particles. The absorbent material may also be substantially free of cellulose fibers, comprising less than 15% by weight of cellulose fibers relative to the total weight of absorbent material, in particular less than 10%, or less than 5% and down to 0% by weight of cellulose fibers. The absorbent material may thus also be free of cellulose fibers, as is known in airfelt-free cores, where the absorbent material consists of, or consists essentially of SAP.

The absorbent core may be relatively thin, in particular thinner than conventional cores comprising cellulosic fibers. In particular, the caliper of the core (before use) as measured at the point corresponding to the crotch point C′, or advantageously at any points of the surface of the core, may be less than about 5.0 mm, in particular from 0.2 mm to 4.0 mm, or from 0.5 mm to 4.0 mm, as measured according to the Thickness Measurement Method described further below.

While the absorbent material may be substantially continuously distributed in the central area, as exemplary illustrated in FIG. 1, the superabsorbent polymers may also be distributed in the central area so that the basis weight of the SAP is advantageously longitudinally profiled so that more absorbency is available towards the crotch portion and the front portion of the central area than in the back portion. The total length of the core wrap is about 10 mm longer in the front and in the back than the central absorbent material portion to provide for sandwich front—and back core wrap end seals.

The absorbent material may be deposited on a substrate to form the central area and the sideline areas by any known processes that allow relatively precise deposition of absorbent material, in particular SAP polymers, advantageously at relatively high speed. For absorbent material comprising cellulose fibers mixed with the SAP polymers, the absorbent material is typically mixed and carried by an air flow within a mixing chamber into a moving mold comprising a 3D insert, the 3D insert being the inverse shape of the desired distribution pattern. Several molds are typically placed on a revolving circular drum to allow for continuous making of the absorbent core. The surface of the mold is in fluid communication with an under-pressure source in the deposition chamber except for the 3D insert (see e.g. EP3,342,386). A second substrate is then used to close the core wrap. The second substrate may be a second portion of the first substrate which is then folded over the deposited absorbent material to finish forming a core wrap, or it may be another substrate layer that is applied over the first substrate to form the absorbent core.

For absorbent material consisting essentially of superabsorbent polymer particles without cellulose fibers, a substantially continuous layer of absorbent material may for example be obtained by assembling two discontinuous absorbent sub-layers in the SAP printing process as disclosed in US2006/024433 (Blessing), US2008/0312617 and US2010/0051166A1 (both to Hundorf et al.). This technique uses a transfer device such as a printing roll to deposit SAP particles onto a substrate disposed on the grid of a support (e.g. a lay-on drum). The printing roll comprises a plurality of cavities arranged in cross-direction lines that can be filled with superabsorbent particles, the position, size and numbers of cavities can be adapted to deposit different quantities of particles according to the desired CD and MD profiles for the absorbent layer. The grid may include a plurality of cross bars extending substantially parallel to and spaced from one another so as to form ribs extending between the cross-bars. The SAP is deposited in the undulations of the substrate inside these ribs. As known in the art indicated above, two such SAP printing roll/laying-on drum systems working in parallel can be used to print twice a SAP layer on two substrates, the substrates being then assembled with the SAP layers in contact with each other thus forming a continuous layer of SAP between the core wrap's top layer and a bottom layer (formed by the two substrates). This SAP printing technology allows high-speed and precise deposition of SAP on a substrate in a desired pattern.

The islands or other shapes of absorbent material may be formed by using SAP printing technology (see e.g. EP2,949,299, Bianchi et al.). The printing roll and the lay-on drum are adapted to provide the desired SAP application pattern, in particular shaping the central area and the sideline areas as desired. Optionally, raised elements on the transfer device may collaborate with corresponding mating strips on the support grid to provide areas free of deposited absorbent material within the central area. The top and bottom layers of the core wrap can be bonded together through some of these material-free areas to form the channel-forming areas 30 discussed below. Thus a SAP printing technique may be advantageously used to make absorbent cores according to the invention. Of course it is not excluded that other manufacturing techniques may be used, or that products are hand-made for research purpose for example.

### Channel-Forming Area 30

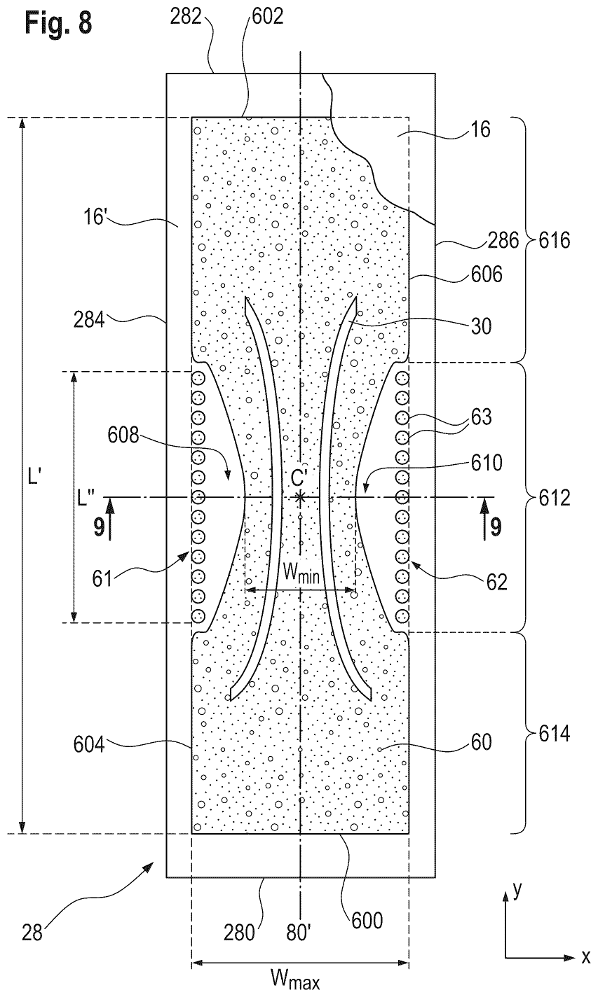

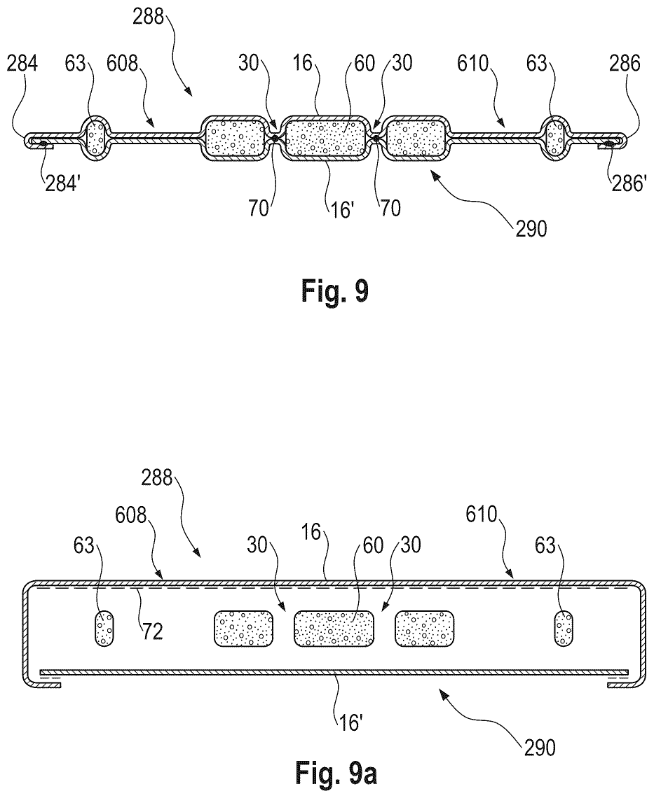

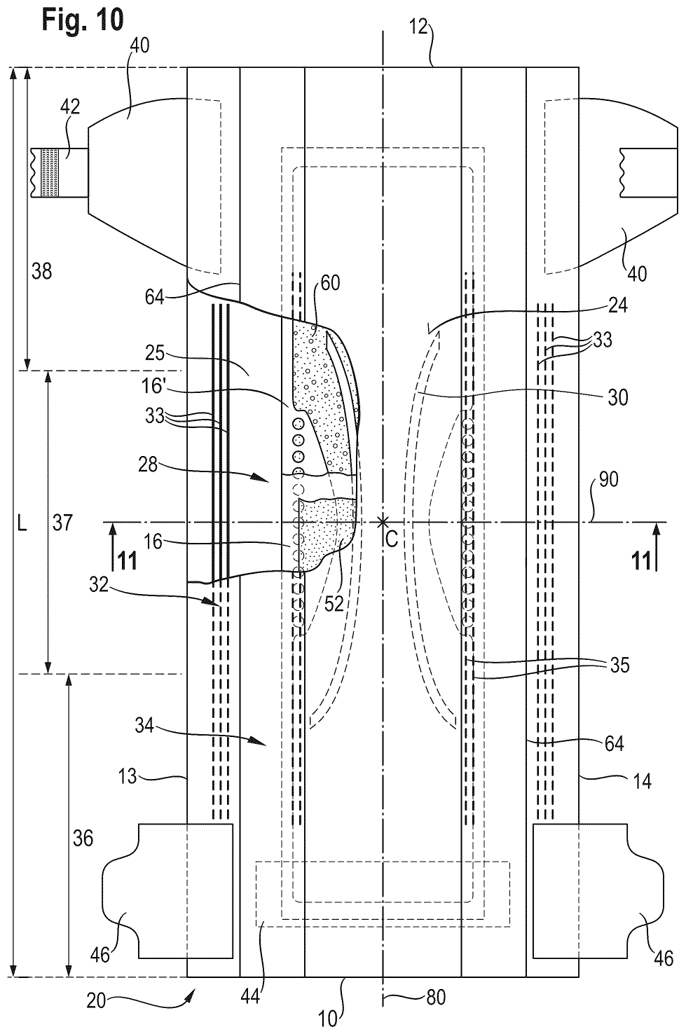

The central area 60 may comprise at least one channel-forming area 30. As illustrated in FIGS. 8-9, for example a pair of channel-forming areas 30 may be disposed symmetrically relative to the longitudinal axis 80′ in the central area 60. A channel-forming area is a macroscopic absorbent material-free area surrounded by absorbent material, through which the top layer 16 of the core wrap is bonded to the bottom layer 16′ of the core wrap. By “substantially free”, it is meant that absorbent material is not deposited on purpose in these areas, but accidental contamination by some absorbent material such as SAP particles during the making process is not excluded. Typically the core wrap bonds 70 between the two layers may be an adhesive bond, or a fusion bond, including ultrasonic bond, or a mechanical bond. During use, the absorbent material surrounding the channel-forming area(s) swells after absorbing a fluid, and the material-free area(s) form three-dimensional channels along the core wrap bonds 70.

The channel-forming areas 30 are typically longitudinally-extending are completely surrounded by absorbent material. The channel-forming areas 30 can also distribute the fluid along the length of the core in the central area, even when the core has already absorbed a fluid due to the core wrap bonds 70. Exemplary disclosures of channel-forming areas in an airfelt-free core can be found in WO2012/170778 (Rosati et al.) and US2012/0312491 (Jackels). Channels and channel-forming areas may of course also be formed in absorbent cores comprising a mix of cellulose fibers and SAP particles. The channel-forming areas 30 may be curved towards the longitudinal axis 80′ as illustrated in FIG. 8. The channel-forming areas may be curved along a smooth curve without inflexion points, as in a couple of inverted brackets: ) (. These channel-forming area(s) may however embody any suitable shapes and any suitable number of channels may be provided. In particular many channel shapes have been suggested in the art. For example, at least a portion of the channel-forming areas may be aligned with the longitudinal axis 80′. In other instances, the absorbent core may be embossed to create the impression of channels.

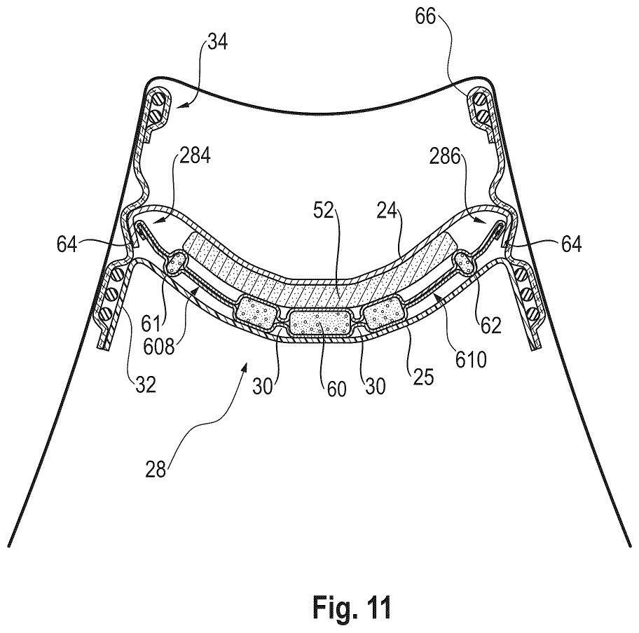

The channel-forming areas 30 can increase the flexibility in the transversal direction of the absorbent core so that the absorbent core can more easily form a three-dimensional basin shape, e.g. as illustrated in FIG. 11, when the article in which it is incorporated is placed between the thighs of a wearer. The width of absorbent material-free areas 30 may be substantially constant along the length of the absorbent material-free areas. The width may also vary along the length of the channel-forming areas. The channel-forming areas may for example have a width of from 1 mm to 20 mm. The channel-forming areas may have a length, as measured in the longitudinal direction, which is at least 10% of the length of the central area 60, typically from 15% to 80% of the length of the central area 60.

As represented in FIG. 9, the channel-forming areas 30 preferably comprise a core wrap bond 70 between the top layer 16 of the core wrap and the bottom layer 16′. This bond 70 may be for example an adhesive bond, a mechanical bond, a fusion bond, an ultrasonic bond or any combinations of these, formed between the top layer and the bottom layer through the absorbent material-free areas 30. The core wrap may also be bonded in other areas of the core in particular to form side seals 284′, 286′ along the longitudinally-extending side edges of the core.

The three-dimensional channels in the absorbent core start forming when the absorbent material starts swelling after absorbing a liquid such as urine. As the core absorbs more liquid, the channels become deeper and more apparent to the eye and the touch. On the other hand, the recesses 608, 610 while also being areas free of absorbent material, may be completely unbonded, or at least not as strongly bonded between the top layer and the bottom layer of the core wrap. This allows the swollen absorbent material to extend sideways from the crotch portion 612 of the absorbent material central area 60. At some point, however the core wrap bond(s) may also be designed to open in a controlled manner when exposed to a large amount of fluid. The core wrap bonds 70 may thus remain substantially intact at least during a first phase as the absorbent material absorbs a moderate quantity of fluid and can expand sideways in the recesses 608, 610. In a second phase, the channel core wrap bonds 70 can start opening to provide more space for the absorbent material to swell while keeping most of the benefits of the channels such as increased flexibility of the core in transversal direction and fluid management. In a third phase, corresponding to a very high saturation of the absorbent core, a more substantial part of the channel bonds can open to provide even more space for the swelling absorbent material to expand. The strength of the core wrap bond within the channels can be controlled for example by varying the amount and nature of the adhesive used for the attaching the two sides of the core wrap. While the recesses and channel bonds 70 may both comprise an auxiliary glue 72 applied on the inner side of the top core wrap layer (and/or bottom core wrap layer) as shown in FIG. 9/9a, the channel bonds 70 may be made stronger by applying localized pressure in the channel-forming areas to adhesively bonds the top and bottom core wrap layers in the channel-forming areas 30.

### Further Components of the Absorbent Core

The absorbent core may comprise one or more layers of glue to help immobilizing the absorbent material, for example as disclosed in US2006/024433 (Blessing), US2008/0312627 and US2010/051166A1 (both to Hundorf et al.) and US2014/027066A1 (Jackels).

The absorbent core may comprise at least one auxiliary glue layer 72 applied on the inner surface of the top layer and/or the bottom layer of the core wrap (shown in the exploded view of FIG. 9a). Such an auxiliary glue is usually a hotmelt glue which forms bonds when cooling. The auxiliary glue may be applied directly over the substrate on which the absorbent material is subsequently deposited, thus at least partially immobilizing the absorbent material on the substrate. The auxiliary glue may also at least partially form a core wrap bond 70 within the channel-forming area(s) 30, if such a bond is desired, especially in combination with pressure being locally applied in the channel-forming area(s) when the auxiliary glue is still warm. The auxiliary glue may also be useful to improve the adhesion of a fibrous thermoplastic material used to immobilize the SAP particles in airfelt-free cores to the substrate. The auxiliary glue can be applied by any adhesive applicator known in the field, in particular bead, slot or spray nozzles. For example, the auxiliary glue can be applied using a slot coating process as a pattern comprising a plurality of spaced-apart slots which may each extend in the longitudinal direction. The slots may for example have a width of from 0.5 mm to 3 mm, and/or have a lateral spacing there-between of from 0.5 mm to 4 mm. The auxiliary glue may be applied along the whole length of absorbent core or intermittently along a shorter length, for example at least in the area of the channel-forming areas are present. The auxiliary glue should be applied over an area aligned with the central area 60 having a width at least equal to Wmin and preferably covering the recesses 608 and 610, optionally up to and including the sideline areas 61, 62 in order to at least partially immobilize the absorbent material in the sideline areas 61, 62 and the central layer 60. The auxiliary glue can also provide some attachment between the core wrap top layer and the core wrap bottom layer in the two recesses 608 and 610, which helps immobilizing the absorbent material. The auxiliary glue bonds in the recesses may be relatively weak and the core wrap layers relatively easily delaminate in the recesses to accommodate the expending superabsorbent material when it absorbs liquid. The channel bond(s) 70 if present may be relatively stronger and remain present during use when the absorbent material swells.

The absorbent core may also comprise a fibrous thermoplastic adhesive material (not shown), also known as microfibrous glue, to help immobilizing the absorbent material within the core wrap, especially for airfelt-free cores. The fibrous thermoplastic adhesive material may be applied, typically by spraying, over an absorbent material that has been discontinuously deposited on a substrate during the core making process, thus forming land and junction areas as indicated above. The fibrous thermoplastic adhesive material contacts the absorbent material and the substrate layer in the absorbent material free junction areas. This imparts an essentially three-dimensional net-like structure to the fibrous layer of thermoplastic adhesive material, which in itself is essentially a two-dimensional structure of relatively small thickness, as compared to the dimension in length and width directions. Thereby, the fibrous thermoplastic adhesive material may provide cavities to cover the absorbent material, and thereby immobilizes this absorbent material. A dual layer core can thus be constructed wherein the land areas of one layer correspond to the material-free junction areas of the other layer and vice versa, resulting in continuous dual absorbent layer.

The adhesive material may advantageously help providing a high immobilization of the absorbent material in dry and wet state. The absorbent core advantageously achieves an SAP loss of no more than about 70%, 60%, 50%, 40%, 30%, 20%, or 10% according to the Wet Immobilization Test described in US2010/051166A1.

Alternative absorbent cores according to the invention are further illustrated in FIGS. 16-19. FIG. 16 illustrates an absorbent having two continuous sideline areas 61,62b separated at each of their extremities from the central area by a gap. FIG. 17 illustrates an absorbent core having two sidelines areas that are connected at their extremities to the central area. FIG. 18 illustrates an absorbent having two continuous sideline areas 61,62b as in FIG. 16, and further comprising a pair of channel-forming areas 30. FIG. 19 illustrates an absorbent core having two sidelines areas that are connected at their extremities to the central area as in FIG. 17 and further comprising a pair of channel-forming areas 30.

Examples of absorbent articles incorporating the absorbent cores according to the invention are illustrated further below.

### Absorbent Article 20

The absorbent cores of the invention may be used in any absorbent articles used to absorb body exudates such a urine from an incontinent individual. The term absorbent articles as used herein include in particular baby and toddler diapers (including training pants), feminine sanitary pads and adult incontinence articles.

An exemplary absorbent article according to the invention is represented in FIG. 5 in the form of a taped diaper 20. FIG. 5 is a top plan view of the exemplary diaper 20, in a flat-out state, with portions of the structure being cut-away to more clearly show the construction of the diaper. This diaper is shown for illustration purpose only. The absorbent article of invention may also be a pant-type article having pre-formed side seams. Unless otherwise indicated, dimensions and areas disclosed herein apply to the article in this flat-out configuration. If some part of the article is under tension due to elasticized components, the article may be typically flattened using clamps along the periphery of the article and/or a sticky surface, so that the topsheet and backsheet can be pulled taut so as to be substantially flat. Closed articles such as diaper pant or training pant may be cut open along the side seams to apply them on a flat surface.

The absorbent article 20 comprises a front edge 10, a back edge 12, and two longitudinally-extending side (lateral) edges 13, 14 joining the front edge and the back edge. The front edge 10 is the edge of the article which is intended to be placed towards the front of the user when worn, and the back edge 12 is the opposite edge. The absorbent article is notionally divided by a longitudinal axis 80 extending from the front edge to the back edge of the article and dividing the article in two substantially symmetrical halves relative to this axis, when viewing the article from the wearer facing side in a flat out configuration, as exemplarily shown in FIG. 5. This axis 80 may typically be concomitant with the longitudinal axis 80′ of the absorbent core 28. The article has a length L as measured along the axis 80 from the back edge to the front edge. The absorbent article 20 can also be notionally divided by a transversal axis 90 into a front region and a back region of equal length measured on the longitudinal axis, when the article is in such a flat state. This article's transversal axis 90 is perpendicular to the longitudinal axis 80 and placed at half the length of the article. The intersection of the longitudinal axis and transversal axis are referred herein as the Crotch Point “C”.

The absorbent article is further notionally divided in a front region, 36, a back region 38 and a crotch region 37 in-between. The front region 36 is defined as the region of the article extending from the front edge 10 and having a length of a third of L along the longitudinal axis 80. The back region 38 is defined as the region of article extending from the back edge 12 of the article and having a length of one third of L along the longitudinal axis 80. The crotch region 37 is the intermediate region between the front and back regions, and also having a length of a third of L along the longitudinal axis 80.

The absorbent article 20 comprises a wearer-facing side, which may be principally formed by a liquid permeable topsheet 24, a garment-facing surface which may be formed by a liquid impermeable backsheet 25, and an absorbent core 28 between the topsheet 24 and the backsheet 25. The absorbent core 28 is shown in isolation in FIG. 1 but of course any absorbent cores according to the invention may be used, for example as shown in FIGS. 8-9 with channel-forming areas The topsheet 24, the backsheet 25, the absorbent core 28 and the other article components may be assembled in a variety of well-known configurations, in particular by gluing and/or heat embossing. Exemplary diaper assemblies are for example generally described in U.S. Pat. Nos. 3,860,003, 5,221,274, 5,554,145, 5,569,234, 5,580,411, and 6,004,306. The absorbent article is preferably thin. In particular, it may have a caliper of from 2.0 mm to 8.0 mm, or from 3.0 mm to 6.0 mm, measured at the crotch point C as measured using the Thickness Measurement Method described below.

### Topsheet 24

Any topsheet known in the art for absorbent articles may be used for the articles of the invention. The topsheet is preferably compliant, soft-feeling, and non-irritating to the wearer's skin. Further, at least a portion of the topsheet is liquid permeable, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. If the topsheet includes fibers, the fibers may be spunbond, carded, wet-laid, meltblown, hydroentangled, or otherwise processed as is known in the art, in particular spunbond PP nonwoven. A suitable topsheet comprising a web of staple-length polypropylene fibers is manufactured by Veratec, Inc., a Division of International Paper Company, of Walpole, MA under the designation P-8. Typical diaper topsheets have a basis weight of from about 10 to about 28 gsm, in particular between from about 12 to about 18 gsm but other basis weights are possible.

Suitable formed film topsheets are also described in U.S. Pat. Nos. 3,929,135, 4,324,246, 4,342,314, 4,463,045, and 5,006,394. Other suitable topsheets may be made in accordance with U.S. Pat. Nos. 4,609,518 and 4,629,643. Such formed films are available from The Procter & Gamble Company of Cincinnati, Ohio as “DRI-WEAVE” and from Tredegar Corporation, based in Richmond, VA, as “CLIFF-T”.

The topsheet may also be treated with a wetting agent to increase its more hydrophilicity. The wetting agent may be a surfactant, as is known in the art. Other possible treatments are for example special coating by nanoparticles, as for example described in U.S. Pat. Nos. 6,645,569, 6,863,933, US2003/148684 and US2005/008839, (Cramer et al.) and U.S. Pat. No. 7,112,621 (Rohrbaugh et al). Any portion of the topsheet may also coated with a lotion as is known in the art. Examples of suitable lotions include those described in U.S. Pat. Nos. 5,607,760, 5,609,587, 5,643,588, 5,968,025 and 6,716,441. The topsheet 24 may also include or be treated with antibacterial agents, some examples of which are disclosed in WO 95/24173. Further, the topsheet, the backsheet or any portion of the topsheet or backsheet may be embossed and/or matte finished to provide a more cloth like appearance.

The topsheet 24 may comprise one or more apertures to case penetration of exudates therethrough, such as urine and/or feces (solid, semi-solid, or liquid). The size of at least the primary aperture is important in achieving the desired waste encapsulation performance. If the primary aperture is too small, the waste may not pass through the aperture, either due to poor alignment of the waste source and the aperture location or due to fecal masses having a diameter greater than the aperture. If the aperture is too large, the area of skin that may be contaminated by “rewet” from the article is increased. Typically, the total area of the apertures at the surface of a diaper may have an area of between about 10 cm² and about 50 cm², in particular between about 15 cm² and 35 cm². Examples of apertured topsheet are disclosed in U.S. Pat. No. 6,632,504. WO 2011/163582 also discloses suitable colored topsheet having a basis weight of from 12 to 18 gsm and comprising a plurality of bonded points. Each of the bonded points has a surface area of from 2 mm² to 5 mm² and the cumulated surface area of the plurality of bonded points is from 10 to 25% of the total surface area of the topsheet.

Although not shown in the drawings, it is possible to bond the topsheet directly or indirectly to the folding guides of the absorbent core. If a liquid management layer is present between the topsheet and the backsheet, the topsheet may also be bonded to or through the folding guide of the liquid management layer. The topsheet may be bonded by any known bonding means, typically adhesive bonding, pressure bonding or heat bonding, or a combination of these. Similarly the topsheet may also be directly or indirectly bonded to at least some of the areas of the core wrap corresponding to the gaps between the winglets of the absorbent core.

### Liquid Management Layers 52, 54

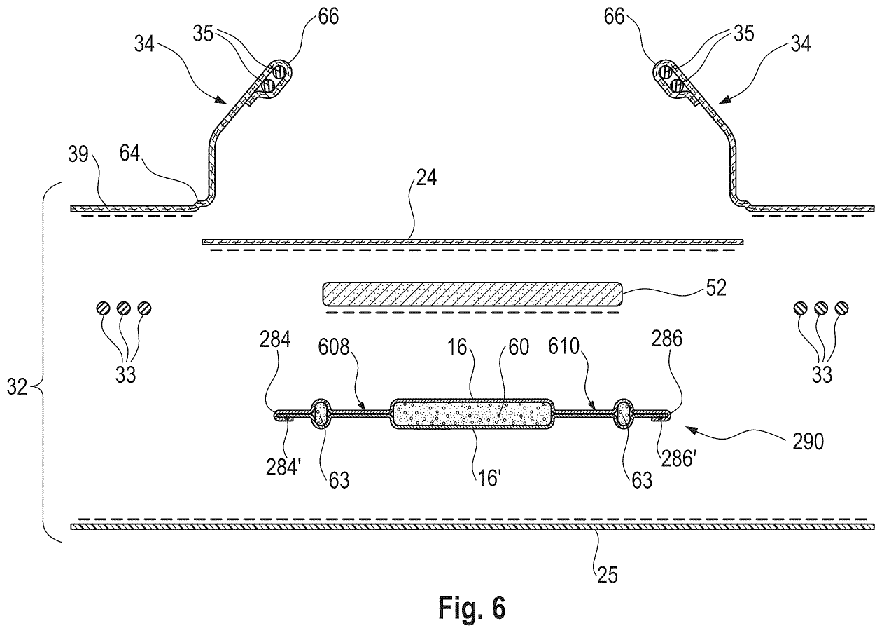

The absorbent article may advantageously comprise at least one liquid management layer 52, also referred herein as acquisition layer, between the topsheet 24 and the absorbent core 28 as illustrated in FIGS. 6-7. Liquid management layers quickly acquire and/or distribute the fluid away from the topsheet and into the core. These liquid management layers are sometimes called “wicking layer”, “surge layer”, “acquisition layer”, “distribution layer” or “acquisition-distribution layer”. Typically, liquid management layers do not comprise SAP, as this may slow the acquisition and distribution of the fluid. The prior art discloses many type of liquid management layer, see for example WO2000/59430 (Daley), WO95/10996 (Richards), U.S. Pat. No. 5,700,254 (McDowall), WO02/067809 (Graef). Liquid management layers are typically placed symmetrically relative to the longitudinal axis of the article.

The liquid management layer 52 may be made of a nonwoven web having hydrophilic properties, often referred to as an acquisition layer. The nonwoven web may be for example provided as a continuous roll of material that is cut according to the desired length and pattern as it is unwound in a converting line. Such an acquisition layer is typically placed directly under the topsheet. Suitable nonwovens are for example through-air bonded (“TAB”) carded nonwovens, resin-bonded (“RB”) carded nonwovens, spunbond or spunlace (hydroentangled) nonwovens. TAB carded nonwovens may for example be made from soft PE/PP bicomponent staple fibers. The air through bonding process locks in loft and compressibility. Resin-bonded carded nonwovens may be made from multi-denier polyester staple fibers (for example: 50/50 or 40/60 mix of 6 denier and 9 denier fibers). Its resilient and open structures are designed to provide excellent fluid acquisition properties. Such acquisition layers are available directly from suppliers, e.g. Fitesa of Simpsonville, South Carolina, USA or TWE Group GmbH, of Emsdetten, Germany. The nonwoven layer may be stabilized by a latex binder for example a styrene-butadiene latex binder (SB latex). Processes for obtaining such latexes are known, for example from EP149,880 (Kwok), US2002/028858 and US2003/0105190 (Dichl). The binder may typically be present in an acquisition layer in excess of about 12%, about 14% or about 16% by weight of the layer. A SB latex is for example commercially available under the trade name GENFLO™ 3160 (OMNOVA Solutions Inc.; Akron, Ohio). Latex bonded acquisition layers are for example further disclosed in US2005/033252A1, US2005/033253A1 or US2005/043694A1 (Schneider). The basis weight of typical acquisition layers ranges from 10 gsm to 200 gsm, in particular 20 gsm to 140 gsm, or 40 gsm to 120 gsm, for example 80 gsm.

Since the function of a liquid management layer is to help transfer the insulting fluid to the absorbent material, it is generally not desirable for the liquid management to extend beyond the edges of the absorbent material area of the core. This can however reduce the fluid handling capacity in conventional articles having a narrow core at crotch. The sideline areas 61, 62 of the invention are disposed in the recesses, transversally outwardly of the central area 60, so that this issue can be avoided. The present invention thus allows using liquid management layer extending transversally beyond the central area 60 in the crotch portion 612 of the central area. The liquid management layer may be typically the same length or shorter in the longitudinal dimension than the absorbent material layer of the absorbent core.

An acquisition layer 52 may be used in the diaper of the invention having a generally rectangular shape and having a width which is larger than Wmin and narrower than or equal to Wmax. Typically the width of the acquisition layer may be slightly less than Wmax, in particular the width of the acquisition layer 52 may be comprised between Wmax and Wmax minus 1 cm. However it is not excluded that a liquid management layer (for example a distribution layer 54 used with an acquisition layer 52, as described below) may have the same or a narrower width than Wmin. For example a liquid management layer may have a similarly shaped non-rectangular outline than the central area 60 at least in the crotch portion 612 of the central area 60, or the liquid management layer may be substantially rectangular with a width which is the same or narrower than Wmin.

Advantageously, the liquid management layer should not hinder or provide a significant resistance to the folding of the core and article in the desired basin shape. The liquid management layer may thus be sufficiently conformable and flexible for this purpose as illustrated in FIG. 7. For example the liquid management layer 52 may be a nonwoven having a basis weight of less than 200 g/m², typically from 30 g/m² to 120 g/m², for example a spunlace nonwoven layer, or an air-through bonded or latex bonded carded layer.

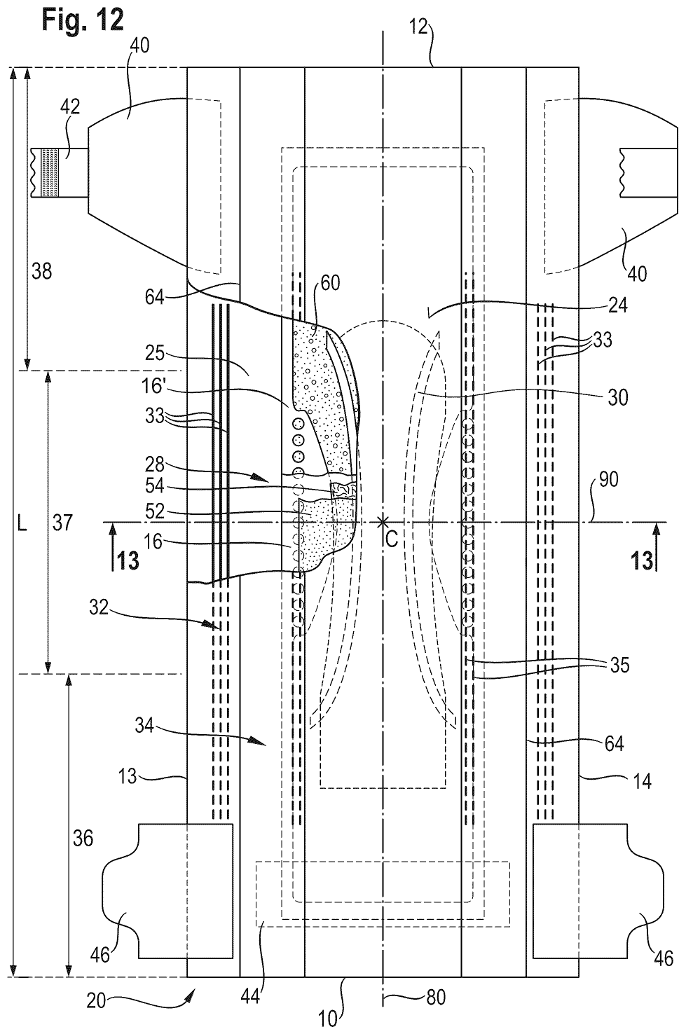

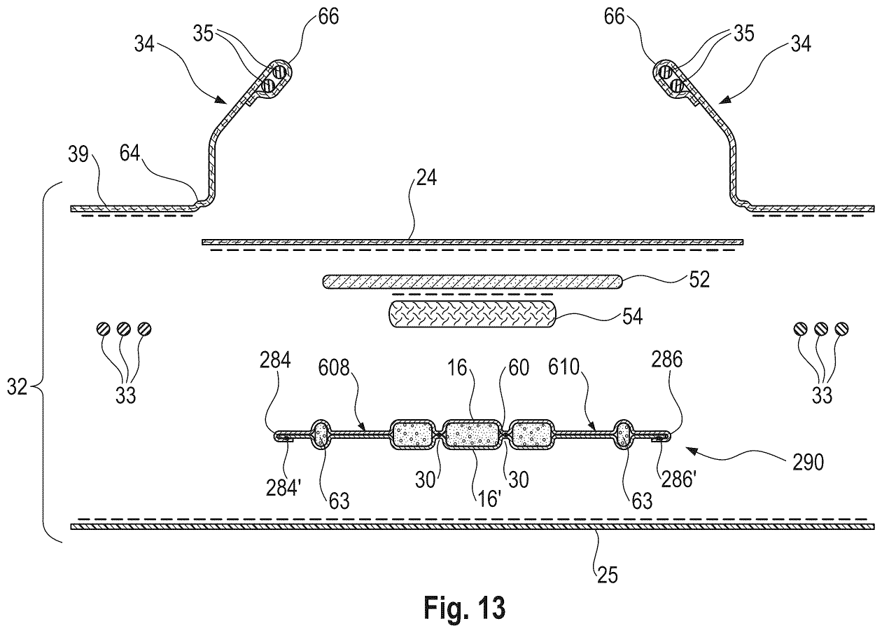

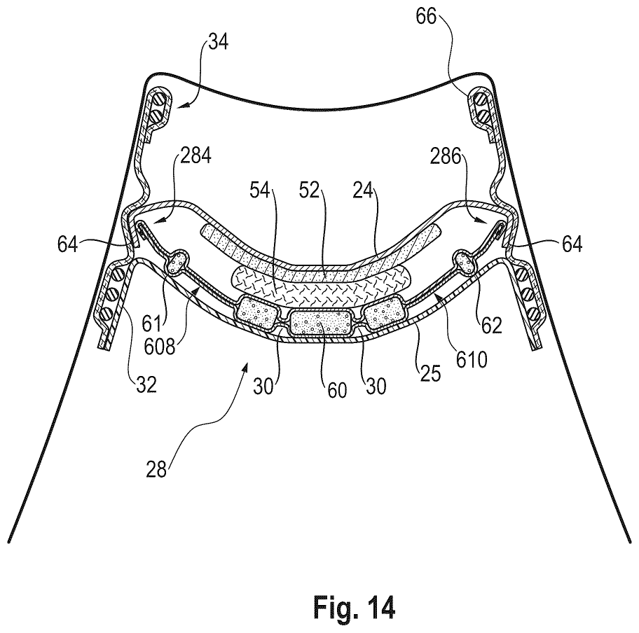

When the absorbent material comprises cellulose fibers mixed with the SAP, a single liquid management layer 52 in the form a hydrophilic nonwoven acquisition layer may be sufficient. Cellulose fibers can typically help acquiring and distributing the fluid within the core. Where the absorbent material of the core is substantially free of cellulose fibers however, it may be advantageous to have two liquid management layers, in the form of the combination of an acquisition layer 52 and a distribution layer 54, as described below and illustrated in any of FIGS. 12-15.

The absorbent articles of the invention may comprise a first and a second liquid management layers. These layers may form an integrated unitary layer or remain discrete layers which are attached to each other for example by adhesive bonding. The article may comprise an acquisition layer 52 directly under the topsheet and a distribution layer 54 between the acquisition layer and the absorbent core, as illustrated in any of FIGS. 12-15. The distribution layer may comprise cross-linked cellulosic fibers and the acquisition layer comprised of a carded, resin-bonded hydrophilic nonwoven. The distribution layer may be profiled in the longitudinal direction and/or have a generally rectangular shaped with a rounded end (“bullet” shaped) towards the back of the diaper. Such dual layer liquid management layers are for example disclosed in further details in WO2014/093323 (Bianchi et al.).

Such a fibrous distribution layer 54 may for example be made on-line by depositing the fibers, for example cross-lined cellulosic fibers, on a forming surface having ridges corresponding to the areas where no fibrous material is desired. Deposition chambers are known wherein a carrier sheet is provided on a forming surface having a series of holes connected to a vacuum, so that the vacuum pulls the fibers in the desired emplacements to form a desired deposited layer. The forming surface of these deposition chambers can be modified to provide a layer of fibrous material having a central portion and side portions separated by folding guides. The fibrous layer is typically formed or transferred on a carrier sheet that should thus have at least the same dimension as a fibrous liquid management layer. The carrier sheet may be the topsheet, another liquid management layer such as a nonwoven acquisition layer 52, or any other layer of the article, for example the core wrap.

The function of a distribution layer is to spread the insulting fluid liquid over a larger surface within the article so that the absorbent capacity of the core can be more efficiently used. Typically, distribution layers can be made of a material comprising synthetic or cellulosic fibers and having a relatively low density. The distribution layer material may be a nonwoven or a fibrous layer comprising unbound or loosely bound hydrophilic fibers, in particular a layer of cross-linked cellulosic fibers. The density of the distribution layer may vary depending on the compression of the article, but may typically range from 0.03 to 0.25 g/cm³, in particular from 0.05 to 0.15 g/cm³ measured at 0.30 psi (2.07 kPa). The distribution layer may also be a material having a water retention value of from 25 to 60, preferably from 30 to 45, measured as indicated in the procedure disclosed in U.S. Pat. No. 5,137,537.

Such a distribution layer 54 may for example comprise at least 50% by weight, optionally consisting of 100%, of cross-linked cellulosic fibers. The cross-linked cellulosic fibers may be crimped, twisted, or curled, or a combination thereof including crimped, twisted, and curled. This type of material has been used in the past in disposable diapers as part of an acquisition system, for example US 2008/0312622 A1 (Hundorf), however not in the manner of the invention. The cross-linked cellulosic fibers provide higher resilience and therefore higher resistance against the compression in the product packaging or in use conditions, e.g. under baby weight. This provides the layer with a higher void volume, permeability and liquid absorption, and hence reduced leakage and improved dryness. The liquid management layer 54 may also be typically profiled so that more material is present at the front and middle part of the article relative to the back of the article. The distribution layer may typically have an average basis weight of from 30 to 400 g/m², in particular from 100 to 300 g/m², with the basis weight varying along the length of the article so that more material is present at the front and middle of the layer than at the back. The liquid management layer may thus be profiled and/or shaped rounded towards the back of the article, as exemplarily disclosed in WO2014/093323 (Bianchi).

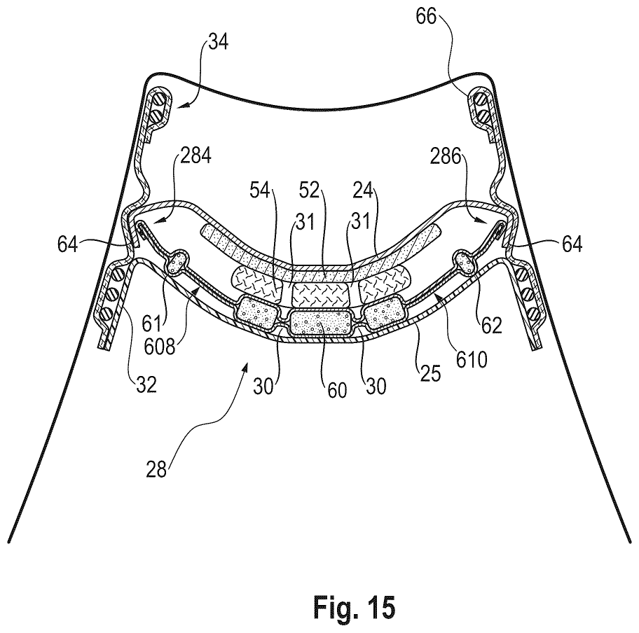

The distribution layer 54 may be provided with folding guides 31 as illustrated in FIG. 15. The folding guides 31 may be at least partially vertically superposed with the channel-forming areas 30 of the absorbent core, when these are present, as illustrated in FIG. 15. Thus, the liquid management layer 54 can easily fold similarly to the core when adapting to a three-dimensional basin shape. “Vertically superposed” means that the position and shape of the folding guides of the liquid management layer vertically correspond to the underlying channel-forming areas of the absorbent core, so that the liquid management layer can readily assume a basin-like shape formed by the underlying absorbent core when the article is put on and worn by the wearer.

The folding guides 31 of the liquid management layer may be superposed with the channel-forming areas 30 of the absorbent core over the whole length of folding guides, but a lower percentage of overlap is also possible. For example, the liquid management layer's folding guides may overlap over at least 50%, 60%, 70% or more of the overall length of the absorbent core's channel-forming area(s). In the remaining areas where there is no overlap, the liquid management layer's folding guides may for example be off-set relative to the absorbent core's channel-forming area(s), or may be shorter and thus not extend to the same length as the absorbent core channel-forming areas 30.

If folding guides 31 are present in such a distribution layer, they may be formed by areas substantially free of the liquid management material, in this case substantially free of unbound or loosely bound hydrophilic fibers such as cross-linked cellulosic fibers as illustrated in FIG. 15.

The invention is however not restricted to this example having two liquid management layers. The majority of articles have in particular for cost reason only one liquid management layer 52, as illustrated in FIG. 6. As indicated previously, there may also be no liquid management layer between the absorbent core and the topsheet, and/or one such layer may be present under the absorbent core, between the absorbent core and the backsheet.

### Backsheet 25

The backsheet 25 may also be made according to any backsheet known in the art for absorbent articles. The backsheet 25 is typically impermeable to liquids (e.g. urine) so that it keeps the garment-facing side of the article dry. The backsheet may for example be or comprise a thin plastic film such as a thermoplastic film having a thickness of less than about 0.10 mm. Exemplary backsheet films include those manufactured by Tredegar Corporation, based in Richmond, VA, and sold under the trade name CPC2 film. Other suitable backsheet materials may include breathable materials which permit vapors to escape from the article while still preventing exudates from passing through the backsheet. A low basis weight nonwoven cover may be attached to the external surface of the film to provide a softer touch.

### Other Components of the Article

The absorbent articles of the invention can comprise any typical components known for the intended purpose of the article. FIGS. 5-7 show other some typical taped diaper components such as a fastening system comprising a pair of back cars 40 extending transversally outwardly from the back edge 12 of the article and each having a fastening tab 42 attached thereto that can fasten to a landing zone 44 placed adjacent the external side of the front edge 10 of the article. These fastening features are typically absent from pant-type articles which have a pre-formed side seam, nevertheless the invention may of course also be used in such pant-types articles. The absorbent article may also comprise other typical components, which are not represented in the Figures, such as a back elastic waist feature, a front elastic waist feature, a transverse barrier element across the topsheet, a wetness indicator between the core and the backsheet that changes appearance when contacted with urine, a lotion application on the topsheet, etc. These components are well-known in the art and will not be further discussed herein. Reference is made to WO2014/093310 where several examples of these components are disclosed in more details.

The absorbent article may however in particular a pair of inner leg cuffs 34 and outer barrier leg cuffs 32, referred together as barrier leg cuffs. The barrier leg cuffs can provide improved containment of liquids and other body exudates approximately at the junction of the torso and legs of the wearer. Typically, the inner cuffs are at least partially formed from a separate material joined to the rest of the article, in particular to the topsheet, but it is not excluded that the inner cuffs can be integral with (i.e. formed from) the topsheet or the backsheet, or any other layer, for example the bottom layer of the core wrap. Typically the material of the barrier leg cuffs may extend through the whole length of the article but is further bonded to the topsheet towards the front edge and back edge of the article so that in these sections the barrier leg cuff material remains flush with the topsheet (tack bonds not shown in FIG. 5 for readability).

The inner leg cuffs each comprises a proximal edge 64 attached to the chassis of the article and a distal edge 66 that can be raised away from the topsheet. The inner leg cuffs 34 can thus protrude away from the topsheet, as shown for example in FIG. 6, at least in the crotch region 37 of the article. The proximal edge 64 may be attached to the topsheet or another layer of the chassis with an intermittent or continuous fusion bond and/or a glue bond. The distal edge 66 of the inner cuffs is elasticized and is designed to fit at the junction of the thighs with the torso of the wearer as illustrated in FIG. 7. Each inner leg cuff 34 typically comprises one, two or more clastic strings 35 close to this free standing distal edge 66. The elastic threads ensure that the inner cuff distal edge is under tension and remain upwards and form a good seal with the thighs of the wearer.

In addition to the inner leg cuffs 34, the article typically comprises outer cuffs 32, which are integrated with the chassis of the absorbent article. The outer cuffs may be at least partially enclosed between the topsheet and the backsheet, or as illustrated in FIG. 6 may be formed by the barrier leg cuff material 39 on one side and the backsheet 25 on the other side. The outer leg cuffs 32 are disposed transversally outwardly relative to the proximal edge 64 of the inner cuffs 34. The outer cuffs 32 provide a better seal around the thighs of the wearer. Usually each outer leg cuff 32 comprises one or more elastic string or elastic element(s) 33 embedded within the chassis of the diaper, for example between the topsheet and backsheet in the area of the leg openings, or as illustrated in FIG. 6 between a barrier leg cuff material 39 and the backsheet. These elastic elements 33, independently or in combination with the elastics 35 of the inner cuffs, may help shaping the absorbent article into a basin when the article is put in place on the wearer. Thus the clastic strings 35 will not only cause the inner cuffs 34 to stand up, and the elastic strings 33 will not only pull the article tighter against the thighs of the wearer, but these inner and outer cuffs will advantageously also pull the sideline areas 61, 62 of the absorbent core upwards. In particular the recesses 608, 610 can function as hinges between the sideline areas 61, 62 and the central area 60. When present, the corresponding areas of a liquid management layer 52 will also stand up to partially form absorbent side walls, as illustrated in FIG. 7.

Various cuff constructions have been disclosed for in the art and may be used in the present invention. U.S. Pat. No. 3,860,003 describes a disposable diaper which provides a contractible leg opening having a side flap and one or more elastic members to provide gasketing cuffs. U.S. Pat. Nos. 4,808,178 and 4,909,803 (Aziz) describe disposable diapers having “stand-up” elasticized flaps (inner leg cuffs) which improve the containment of the leg regions. U.S. Pat. No. 4,695,278 (Lawson) and U.S. Pat. No. 4,795,454 (Dragoo) describe disposable diapers having dual cuffs, including outer leg cuffs and inner leg cuffs. More recently, WO2005/105010 (Ashton) discloses a dual cuff system made of a continuous cuff material.

The combined elastic forces provided by the different elasticized components of the article may thus bring or facilitate bringing the article into a basin shape when the article is placed on a wearer. Although not represented, the article of the invention may further comprise other longitudinally-extending elasticized elements as known in the prior art.

More generally, adjacent layers within the article will be joined together using conventional bonding method such as adhesive coating via slot coating, spiral gluing, or spraying on the whole or part of the surface of the layer, or thermo-bonding, or pressure bonding or combinations thereof. Most of the bonding between components is for clarity and readability not represented in the Figure. Bonding between the layers of the article should be considered to be present unless specifically excluded. Adhesives may be typically used to improve the adhesion of the different layers. For example, the backsheet and the core wrap may be glued using a core-to-backsheet gluing pattern as disclosed in WO2012/170341A1 (Hippe), or a full coverage pattern using several spiral glue applicators. If for example the backsheet is attached by gluing or otherwise to the areas of the core wrap corresponding to the folding guides (not shown), the folding guides may become more visible to the user from the garment-facing side of the article. Any typical hotmelt adhesives may be used. It is also possible to use a printed adhesive layer, for example between the topsheet and absorbent core or liquid management layer, which may be optionally visible through the topsheet, as exemplary disclosed in WO2014/078247.

Typically, adjacent layers and components of the absorbent article will be joined together using conventional bonding method such as adhesive coating (via slot coating, spiral gluing, or spraying) on the whole or part of the surface of the layer, or thermo-bonding, or pressure bonding or combinations thereof. Most of the bonding between components is for clarity and readability not represented in the Figure. Bonding between the layers of the article should be considered to be present unless specifically excluded. Adhesives may be typically used to improve the adhesion of the different layers, for example between the backsheet and the core wrap as disclosed in WO2012/170341A1. The adhesives used may be any standard hotmelt glue as known in the art. FIG. 6 shows in dotted lines the presence of adhesive between selected components, the surface being with the dotted lines disposed closest indicating where the adhesive is first applied before the components are brought in contact.

### Packaging

The absorbent articles of the present disclosure may be placed into packages. The packages may comprise polymeric films and/or natural fibers and/or other materials. The package material may comprise paper, paper with a barrier layer, or a paper/film laminate. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

The absorbent articles may be packaged in any type of conventional packaging. The absorbent articles may be in particular compressed when packaged to save space. The package may thus comprise a plurality of bi-folded absorbent articles, wherein the articles in the package have an in-bag stack height of less than about 80 mm, according to the In-Bag Stack Height Test as described in WO2011/041352 (Weisman et al.), incorporated herein by reference. The packaged absorbent articles may for example have an in-bag stack height of from about 72 mm to about 80 mm or from about 74 mm to about 78 mm, specifically reciting all 0.5 mm increments within the specified ranges and all ranges formed therein or thereby, according to the In-Back Stack Height Test. The method used to measure the In-Bag Stack Height is described in further details in WO2011/041352 (Weisman) with the Universal Diaper Packaging Tester illustrated in FIG. 19 of WO2008/155702A1 (Hundorf).

Many absorbent articles are bi-folded along their transversal axis 90 when packed in their bags. When the articles are highly compressed in the bag to save space, this may cause a permanent fold line to appear along the bi-fold line of the articles, depending on the material used and the storage time of the articles in bag. Thus it is also considered that the articles may be packaged under a lower compression to avoid this issue, for example corresponding to an in-bag stack height above 80 mm, in particular between 84 mm and 120 mm. The articles may also be packaged tri-folded, as exemplarily disclosed in WO2008/155702 (Hundorf).

### EXAMPLES

The present invention is applicable to a wide range of absorbent cores and absorbent articles. The dimensions and materials used may vary depending on the intended applications as is known in the art. For example, absorbent cores for larger size diapers will typically be longer and larger and comprise more absorbent material than absorbent cores for smaller size diapers. The following illustrates dimensions and material for an exemplary core comprising a pair of channel-forming areas similar to the one represented in FIG. 8. The following are exemplary dimension values for an absorbent core suitable for a “size 4” (“Midi”) diaper, which can be recommended for baby weighing between 7 kg and 18 kg.
L′=400 mm
L″=150 mm
616=140 mm (length)
612=160 mm (length)
614=100 mm (length)
Wmax=110 mm
Wmin=76 mm
Number of islands 63 on each sideline area 61, 62=15
Diameter of each island 63=5-8 mm
Gap between each island 63=2-5 mm
Total area L′×Wmax=44,000 mm²
Combined area of recesses 608 and 610 (including islands)=4.600 mm² (representing 10.5% of the rectangle defined by L′×Wmax)
Channel 30 Length (in y direction)=227 mm
Channel 30 Width=8 mm
Minimum Distance Channel-forming area to Recess=20 mm
Minimum Distance Between Channel-forming areas=20 mm

In this example, the core wrap comprised a top layer C-wrapped around a bottom layer with adhesive longitudinal seals (as in FIG. 2). The top layer had a width of 165 mm, the bottom layer had a width of 130 mm. The final core bag width was approximately 120 mm. The top layer was a 8 gsm water permeable propylene spunbonded nonwoven (from Fitesa Germany) and the bottom layer an hydrophobic 10 gsm nonwoven (from Fibertex). The core wrap was sealed by gluing at its font end and its back end. The absorbent material was SAP particles, L805, ex Nippon Shokubai Group, which were deposited for one half on the top layer and the other half on the bottom layer of the core wrap and immobilized by a microfibrous glue and an auxiliary slot glue layer on one side of the core wrap (as indicated above). The total amount of SAP was 11 g. The basis weight of the SAP in the central area is advantageously longitudinally profiled so that more absorbency is available towards the middle and the front of the core. The total length of the core wrap was about 10 mm longer in the front and in the back than the central absorbent material portion to provide for sandwich front and back core wrap end seals.

The absorbent core above was incorporated in a Size 4 diaper chassis similar to commercially available Pamper Swaddlers Size 4, comprising a dual acquisition distribution layer comprising a nonwoven acquisition layer 52 and a distribution layer 54 (as illustrated in FIG. 12) as described above. The width of the rectangular acquisition layer 52 was in this example 105 mm and the distribution layer was shaped with a narrow crotch width (80-68-80 mm) and a rounded bullet end towards the back of the diaper.

### Comparative Examples

As comparison, commercially available Pampers Swaddlers Size 4 having a rectangular absorbent material area including a pair of curved channel-forming areas. The channel-forming areas, the SAP amount and provenance, and the acquisition layer were as in the invention example. The distribution layer had constant width of 80 mm with a bullet shape towards the back of the diaper.

### Testing:

Both diapers were put on an agile mannequin having a fluid inlet in the crotch allowing two gushes of 75 ml saline solution (total 150 ml) with 5 minutes of controlled simulated motion after each gush. The vertical position (sagging) and width of the crotch portion of the diaper were then measured. The measurements were repeated at least 5 times for each products.

### Results:

The inventive diaper had a vertical position on average 5 mm higher than for the control marketed diapers. Similarly, the average width of the inventive diaper was 58 mm vs 68 mm for the marketed diapers. These results were statistically significantly different, and show that the inventive diapers have a better overall wet fit (less sagging and side expansion) than the control diapers.

### Test Procedures

The values indicated herein are measured according to the methods indicated herein below, unless specified otherwise. All measurements are performed at 21° C.±2° C. and 50%±20% RH, unless specified otherwise. All samples should be kept at least 24 hours in these conditions to equilibrate before conducting the tests, unless indicated otherwise. All measurements should be reproduced on at least 4 samples and the average value obtained indicated, unless otherwise indicated.

### Centrifuge Retention Capacity (CRC)

The CRC measures the liquid absorbed by the superabsorbent polymer particles for free swelling in excess liquid. The CRC is measured according to EDANA method WSP 241.2-05.

### Urine Permeability Measurement (UPM) Test Method

This method is used to determine the permeability of a swollen hydrogel layer. The results are generally expressed in UPM units equal to 1×10⁻⁷ cm³·s/g. The Urine Permeability Measurement Test is disclosed in PCT application WO2012/174026A1, incorporated herein by reference.

### Thickness Measurement Method

This method is used to measure the thickness of a component of an article, or of the article (“sample”) itself in a standardized manner.

Equipment: Mitutoyo manual caliper gauge with a resolution of 0.01 mm, or equivalent instrument.

Contact Foot: Flat circular foot with a diameter of 17.0 mm (±0.2 mm). A circular weight may be applied to the foot (e.g., a weight with a slot to facilitate application around the instrument shaft) to achieve the target weight. The total weight of foot and added weight (including shaft) is selected to provide 4.14 kPa of pressure to the sample.

The caliper gauge is mounted with the lower surface of the contact foot in an horizontal plane so that the lower surface of the contact foot contacts the center of the flat horizontal upper surface of a base plate approximately 20 cm×25 cm. The gauge is set to read zero with the contact foot resting on the base plate.

Ruler: Calibrated metal ruler graduated in mm.

Stopwatch: Accuracy 1 second.

Sample preparation: The sample is conditioned at least 24 hours as indicated above.

Measurement procedure: The sample is laid flat with the bottom side, i.e. the side intended to be placed away from the wearer facing down. The point of measurement (if not otherwise indicated the middle of the sample) is carefully drawn on the top side of the sample, taking care not to compress or deform the sample.

The contact foot of the caliper gauge is raised and the sample is placed flat on the base plate of the caliper gauge with the top side of the sample up so that when lowered, the center of the foot is on the marked measuring point.

The foot is gently lowered onto the sample and released (ensure calibration to “0” prior to the start of the measurement). The caliper value is read to the nearest 0.01 mm, 10 seconds after the foot is released.

The procedure is repeated for each sample. Ten samples are measured in this manner for a given material and the average caliper is calculated and reported with an accuracy of one tenth mm.

### General

Dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent core extending in a longitudinal direction and a transversal direction, the absorbent core having a longitudinal axis, the absorbent core comprising an absorbent material and a core wrap, wherein the absorbent material is between a top layer and a bottom layer of the core wrap, the absorbent material comprising superabsorbent polymer particles and optionally cellulose fibers, wherein the absorbent material defines within the core wrap an absorbent material area that comprises:
a central area comprising a front edge, a back edge, two longitudinally-extending side edges joining the front edge to the back edge, and a length measured along the longitudinal axis,
wherein the central area comprises a first recess and a second recess disposed symmetrically relative to the longitudinal axis at a position between the front edge and the back edge, these recesses delimiting a crotch portion of the central area having a smaller width than the maximum width of the central area;
a first sideline area in the first recess, and a second sideline area in the second recess, wherein each sideline area has a length which is from about 25% to about 70% of the length of the central area.

2. The absorbent core according to claim 1, wherein the width of the crotch portion of the central area is smaller than the width at least one of the front edge and the back edge of the central area.

3. The absorbent core according to claim 1, wherein the first sideline area and the second sideline area each comprises a plurality of islands of absorbent material, each island being separated from adjacent islands by an absorbent material-free gap, when the absorbent core is considered in a flat state.

4. The absorbent core according to claim 1, wherein each of the first sideline area and the second sideline area comprises from two to twenty islands of absorbent material.

5. The absorbent core according to claim 1, wherein the islands of the absorbent material of each sideline area are substantially longitudinally-aligned.

6. The absorbent core according to the preceding claim, wherein the plurality of islands for each sideline are longitudinally-aligned parallel to the longitudinal direction.

7. The absorbent core according to claim 1, wherein the central area comprises at least one channel-forming area, which is an area free of absorbent material through which the top layer of the core wrap is bonded to the bottom layer of the core wrap, so that when the absorbent material surrounding the channel-forming area swells, a three-dimensional channel is formed along the channel bond.

8. The absorbent core according to claim 1, comprising an auxiliary glue between the top layer core wrap and the absorbent material and/or between the absorbent material and the bottom layer core wrap.

9. The absorbent core according to claim 1, wherein the superabsorbent polymer particles are at least partially immobilized within the core wrap by a microfibrous glue.

10. The absorbent core according to claim 1, wherein the first recess and the second recess have a combined area in the range of from 5% to 30% of the area of a trapezoid or rectangle defined by the front edge and the back edge of the central area.

11. An absorbent article for personal hygiene comprising a chassis, the chassis comprising a liquid-permeable topsheet on the article's wearer-facing side, a liquid-impermeable backsheet on the article's garment-facing side, and an absorbent core according to claim 1 between the topsheet and the backsheet.

12. The absorbent article according to claim 11, comprising a liquid management layer between the topsheet and the absorbent core, wherein the liquid management layer does not extend widthwise beyond the absorbent material area comprising the central area and the sidelines areas.

13. The absorbent article according to claim 11, comprising a distribution layer between the topsheet and the liquid management layer.

14. The absorbent article according to claim 13, wherein the distribution layer comprises at least one folding guide, wherein the folding guide is at least partially superposed with the at least one channel-forming area of the absorbent core.

15. The absorbent article according to claim 10, the absorbent article comprising a longitudinal axis, a front region, a back region and an intermediate crotch region, each region measuring a third of the length of the article as measured along the longitudinal axis of the article, and wherein the central area of the absorbent core extends longitudinally across the front region, crotch region and back region of the absorbent article, and the first and second sideline areas of the absorbent core are at least partially within the crotch region of the article.

16. The absorbent article according to claim 10, wherein the absorbent article further comprises a pair of elasticized inner leg cuffs, each inner leg cuff having a proximal edge attached to the chassis, and a free-standing elasticized distal edge;
a pair of elasticized outer cuffs;
wherein at least one of the one or more elasticized inner and outer cuffs exert a contraction force on the chassis of the article so that the absorbent core takes a basin shape when the article is placed on a wearer.

17. An absorbent article for personal hygiene comprising a chassis, the chassis comprising a liquid-permeable topsheet on the article's wearer-facing side, a liquid-impermeable backsheet on the article's garment-facing side, and an absorbent core;
wherein the absorbent core extends in a longitudinal direction and a transversal direction, the absorbent core having a longitudinal axis, the absorbent core comprising an absorbent material and a core wrap, wherein the absorbent material is between a top layer and a bottom layer of the core wrap, the absorbent material comprising superabsorbent polymer particles and cellulose fibers, wherein the absorbent material defines within the core wrap an absorbent material area that comprises:
a central area comprising a front edge, a back edge, two longitudinally-extending side edges joining the front edge to the back edge, and a length measured along the longitudinal axis;
wherein the central area comprises a first recess and a second recess disposed symmetrically relative to the longitudinal axis at a position between the front edge and the back edge, these recesses delimiting a crotch portion of the central area having a smaller width than the maximum width of the central area, in particular a smaller width than at least one of the front edge and the back edge; and
a first sideline area present in the first recess, and a second sideline area present in the second recess, wherein each sideline area has a length which is from about 25% to about 70% of the length of the central area.

18. An absorbent article for personal hygiene comprising a chassis, the chassis comprising a liquid-permeable topsheet on the article's wearer-facing side, a liquid-impermeable backsheet on the article's garment-facing side, and an absorbent core;
wherein the absorbent core extends in a longitudinal direction and a transversal direction, the absorbent core having a longitudinal axis, the absorbent core comprising an absorbent material and a core wrap, wherein the absorbent material is between a top layer and a bottom layer of the core wrap, the absorbent material comprising superabsorbent polymer particles and being free of cellulose fibers, wherein the absorbent material defines within the core wrap an absorbent material area that comprises:
a central area comprising a front edge, a back edge, two longitudinally-extending side edges joining the front edge to the back edge, and a length measured along the longitudinal axis;
wherein the central area comprises a first recess and a second recess disposed symmetrically relative to the longitudinal axis at a position between the front edge and the back edge, these recesses delimiting a crotch portion of the central area having a smaller width than the maximum width of the central area; and
a first sideline area in the first recess, and a second sideline area in the second recess, wherein each sideline area has a length which is from about 25% to about 70% of the length of the central area.

19. The absorbent article according to claim 18, wherein the superabsorbent polymer particles are at least partially immobilized within the core wrap by a microfibrous glue.

20. The absorbent article according to claim 18, comprising a liquid management layer between the topsheet and the absorbent core, wherein the liquid management layer does not extend widthwise beyond the absorbent material area comprising the central area and the sidelines areas; and a distribution layer between the topsheet and the liquid management layer.
